(19) United States
(12) Patent Application Publication
(10) Pub. No.: US 2023/0173086 A1
(43) Pub. Date: Jun. 8, 2023

(54) PEPTIDE CONJUGATES AND THERAPEUTIC AGENTS HAVING IMPROVED PHARMACOLOGICAL AND PHARMACOKINETIC PROPERTIES

(71) Applicant: Resolute Bio, Inc., Beverly, MA (US)

(72) Inventors: Bradley L. Pentelute, Revere, MA (US); Tomi K. Sawyer, Southborough, MA (US); Dinara S. Gunasekera, Cambridge, MA (US); Solimar G. Santiago, Medford, MA (US); Jonathon R. Sawyer, Beverly, MA (US)

(21) Appl. No.: 17/969,289

(22) Filed: Oct. 19, 2022

Related U.S. Application Data
(60) Provisional application No. 63/257,294, filed on Oct. 19, 2021.

(51) Int. Cl. A61K 47/68; A61K 47/64; A61K 47/54

(52) U.S. Cl. CPC A61K 47/6811; A61K 47/645; A61K 47/542; A61K 47/6845

(57) ABSTRACT

Disclosed are conjugates, comprising a peptide moiety, a linker, and an active moiety. Also disclosed are methods of using the conjugates for treating a disease, inducing antigen-specific immunotolerance to the active moiety, or extending the half-life of the active moiety.

### RELATED APPLICATION

This application claims the benefit of priority to U.S. Provisional Patent Application No. 63/257,294, filed Oct. 19, 2021.

### BACKGROUND

Erythrocytes, with a lifespan of 120 days, transport oxygen and are the primary component of blood. The covalent or non-covalent attachment of antigens or therapeutics (e.g., peptides, proteins, enzymes, antibodies) onto erythrocytes can improve their therapeutic properties by increasing half-life and promoting antigen or drug-specific tolerance. Consequently, anti-drug antibodies and inflammatory responses are reduced.

### SUMMARY

One aspect of the invention provides conjugates, compositions, and methods useful for inducing immune tolerance and/or half-life extension.

Accordingly, provided herein is a conjugate of Formula I, comprising a peptide moiety, a linker, and an active moiety:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)

wherein

R_{N} is the terminal H₂N— group of Xₐₐ¹;

Xₐₐ¹ is an optionally substituted D-Phe;

Xₐₐ² is an optionally substituted D-amino acid residue selected from D-Phe, D-Trp, D-Tyr, and D-Ala;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Ala, D-Phe, D-His, D-Lys, D-Met, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-amino acid residue selected from D-Lys and D-His;

R_{C} is the C-terminal —C(O)—NH— group of Xₐₐ¹⁰;

Y is the linker which links the peptide moiety and the active moiety; and

Z is the active moiety.

Another aspect of the invention relates to methods of treating or preventing a disease in a subject in need thereof comprising administering to the subject an effective amount of a conjugate of Formula (I).

A further aspect of the invention relates to a method of treating or preventing diabetes in a subject in need thereof comprising administering to the subject an effective amount of a conjugate of Formula (I).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1: LCMS characterization of purified Peptide Dimer 1. The purified sample was analyzed using Agilent Zorbax 300SB—C18, 5 μm, 2.1×150 mm column and gradient of 5-65% B over 20 minutes (A: 0.1% formic acid in water, B: 0.1% formic acid in acetonitrile, 0.8 mL/min, 40° C.).

FIG. 2: Mouse red-blood cell binding (RBC) of Peptide Dimer 1, which associated with the surface of RBCs in a concentration-dependent manner.

FIG. 3: LCMS characterization of purified Peptide Dimer 2. The purified sample was analyzed using Agilent Zorbax 300SB—C18, 5 μm, 2.1×150 mm column and gradient of 20-80% B over 20 minutes (A: 0.1% formic acid in water, B: 0.1% formic acid in acetonitrile, 0.8 mL/min, 40° C.).

FIG. 4: LCMS characterization of purified Intermediate Dimer 3. The purified sample was analyzed using Agilent Zorbax 300SB—C18, 5 μm, 2.1×150 mm column and gradient of 20-80% B over 20 minutes (A: 0.1% formic acid in water, B: 0.1% formic acid in acetonitrile, 0.8 mL/min, 40° C.).

FIG. 5: LCMS characterization of purified Peptide Dimer 4. The purified sample was analyzed using Agilent Zorbax 300SB—C18, 5 μm, 2.1×150 mm column and gradient of 20-80% B over 20 minutes (A: 0.1% formic acid in water, B: 0.1% formic acid in acetonitrile, 0.8 mL/min, 40° C.).

FIG. 6: Mass spectrometry analysis of the crude reaction mixture indicates that up to 4 copies of the peptide intermediate 5 was covalently added to adalimumab. An Agilent 6545 Q-TOF mass spectrometer coupled to a 1260 LC was used for analysis (gradient: 5-80% over 12 min, 0.8 mL/min, 60° C.) using a NanoChrom Technologies ChromGel RP-300 5 μm, 3.0×150 mm column.

FIG. 7: Mouse red-blood cell binding (RBC) of antibody-peptide conjugate 6, which associated with the surface of RBCs in a concentration-dependent manner.

### DETAILED DESCRIPTION

Disclosed herein are erythrocyte-binding conjugates comprising a D-peptide moiety, a linker, and an active moiety which exhibit superior pharmacological and pharmacokinetic properties relative to L-peptide conjugates. The D-peptide moiety provides selective, high affinity binding to erythrocytes.

The disclosed conjugates comprising an antigen moiety as the active moiety are useful for inducing antigen-specific immunotolerance in a subject in need thereof. The conjugate directs the antigen to mitigate an antibody response to the antigen.

The disclosed conjugates comprising a drug moiety as the active moiety are useful for treating a disease in a subject in need thereof, wherein the disease is the therapeutic target of the drug. The conjugates disclosed herein, when administed to the subject, also extends the half-life of the linked drug as compared to administration of the free drug.

The disclosed conjugates comprising a GLP-1 agonist moiety as the active moiety are useful for treating a disease in a subject in need thereof, wherein the disease is the therapeutic target of the GLP-1 agonist. The conjugates disclosed herein, when administed to the subject, also extends the half-life of the linked GLP-1 agonist as compared to administration of the free GLP-1 agonist.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, compounds of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon—carbon double bond may be in an E (substituents are on opposite sides of the carbon—carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl goups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example, from 2 to 12 carbon atoms, which contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to a cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

“Ureido” refers to an optionally substituted urea moiety, e.g., —NHC(O)NH2 or —NHC(O)NHR, wherein R is alkyl, aryl, aralkyl, heterocyclyl, heteroaryl, or heteroarylalkyl.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter ascompared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

The term “diabetes and related diseases or related conditions” refers, without limitation, to Type II diabetes, Type I diabetes, impaired glucose tolerance, obesity, hyperglycemia, Syndrome X, dysmetabolic syndrome, diabetic complications, and hyperinsulinemia.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a conjugate, comprising a peptide moiety, a linker, and an active moiety, having the following structure of formula (I):

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)

wherein

R_{N} is the terminal H₂N— group of Xₐₐ¹;

Xₐₐ¹ is an optionally substituted D-Phe;

Xₐₐ² is an optionally substituted D-amino acid residue selected from D-Phe, D-Trp, D-Tyr, and D-Ala;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Ala, D-Phe, D-His, D-Lys, D-Met, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-amino acid residue selected from D-Lys and D-His;

R_{C} is the C-terminal —C(O)—NH— group of Xₐₐ¹⁰;

Y is the linker which links the peptide moiety and the active moiety; and

Z is the active moiety.

In certain embodiments, Z is a drug moiety. In other embodiments, Z is a GLP-1 agonist moiety. In other embodiments, Z is an erythrocyte-targeted antigen moiety.

In certain embodiments,

R_{N} is the terminal H₂N— group of Xₐₐ¹;

Xₐₐ¹ is an optionally substituted D-Phe;

Xₐₐ² is an optionally substituted D-amino acid residue selected from D-Phe, D-Trp and D-Tyr;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Phe, D-His, D-Lys, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-Lys;

R_{C} is the C-terminal —C(O)—NH— group of Xₐₐ¹⁰;

Y is the linker which links the peptide moiety and the active moiety; and

Z is the active moiety.

In certain embodiments,

R_{N} is the terminal H₂N— group of Xₐₐ¹;

Xₐₐ¹ is an optionally substituted D-Phe;

Xₐₐ² is an optionally substituted D-amino acid residue selected from D-Phe, D-Trp and D-Tyr;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro;

Xₐₐ⁵ is an optionally substituted D-amino acid residue selected from D-Phe, D-His, D-Leu, D-Met, D-Pro, D-Ala, N-Me-D-Ala, D-Val, D-Trp, and D-Tyr;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Phe, D-His, D-Lys, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, D-Ser, D-Glu, D-Thr, D-Val, D-Trp, and D-Tyr;

Xₐₐ¹⁰ is an optionally substituted D-Lys;

R_{C} is the C-terminal —C(O)—NH— group of Xₐₐ¹⁰;

Y is the linker which links the peptide moiety and the active moiety; and

Z is the active moiety.

In certain embodiments,

Xₐₐ³ is an optionally substituted D-amino acid residue selected from D-His, D-Leu, D-Met, D-Val, and D-Tyr;

Xₐₐ⁵ is an optionally substituted D-amino acid residue selected from D-Phe, D-His, D-Leu, D-Met, D-Pro, D-Val, D-Trp, and D-Tyr;

Xₐₐ⁶ is an optionally substituted D-amino acid residue selected from D-Ala, D-Phe, D-Leu, D-Met, D-Pro, D-Ser, D-Thr, D-Val, D-Trp, and D-Tyr;

Xₐₐ⁷ is an optionally substituted D-amino acid residue selected from D-Ala, D-Phe, D-Leu, D-Met, D-Trp, and D-Tyr; and

Xₐₐ⁹ is an optionally substituted D-amino acid residue selected from D-Phe, D-His, D-Leu, D-Pro, D-Ser, D-Glu, D-Thr, D-Val, D-Trp, and D-Tyr.

In certain embodiments,

Xₐₐ⁵ is an optionally substituted D-amino acid residue selected from D-Phe, D-His, D-Leu, D-Met, D-Pro, D-Ala, D-Val, D-Trp, and D-Tyr;

Xₐₐ⁶ is an optionally substituted D-amino acid residue selected from D-Ala, D-Phe, D-Leu, D-Met, D-Nle, D-HNle, D-Abu, D-Cys, D-Pro, D-Ser, D-Thr, D-Val, D-Trp, and D-Tyr; and

Xₐₐ⁹ is an optionally substituted D-amino acid residue selected from D-Phe, D-His, D-Leu, D-Pro, D-Ala, D-Ser, D-Glu, D-Thr, D-Val, D-Trp, and D-Tyr;

In certain embodiments, Xₐₐ¹ is unsubstituted D-Phe.

In certain embodiments, Xₐₐ² is an unsubstituted D-amino acid residue selected from D-Phe, D-Trp and D-Tyr.

In certain embodiments, Xₐₐ² is D-Trp.

In certain embodiments, Xₐₐ² is D-Tyr.

In certain embodiments, Xₐₐ³ is an unsubstituted D-amino acid residue selected from D-His, D-Leu, D-Met, D-Val, and D-Tyr.

In certain embodiments, Xₐₐ³ is D-His.

In certain embodiments, Xₐₐ⁴ is D-Pro.

In certain embodiments, Xₐₐ⁵ is an unsubstituted D-amino acid residue selected from D-Phe, D-His, D-Leu, D-Met, D-Pro, D-Val, D-Trp, and D-Tyr.

In certain embodiments, Xₐₐ² is D-Phe.

In certain embodiments, Xₐₐ² is D-Trp.

In certain embodiments, Xₐₐ⁶ is an unsubstituted D-amino acid residue selected from D-Ala, D-Phe, D-Leu, D-Met, D-Pro, D-Ser, D-Thr, D-Val, D-Trp, and D-Tyr.

In certain embodiments, Xₐₐ⁶ is selected from D-Ala, D-Thr, and D-Ser.

In certain embodiments, Xₐₐ⁷ is an unsubstituted D-amino acid residue selected from D-Ala, D-Phe, D-Leu, D-Met, D-Trp, and D-Tyr.

In certain embodiments, Xₐₐ⁷ is selected from D-Met and D-Trp.

In certain embodiments, Xₐₐ⁸ is an unsubstituted D-amino acid residue selected from D-Phe, D-His, D-Lys, D-Leu, D-Met, D-Glu, D-Thr, D-Val, D-Trp, and D-Tyr.

In certain embodiments, Xₐₐ⁸ is an unsubstituted D-amino acid residue selected from D-Phe, D-His, D-Lys, D-Leu, D-Glu, D-Thr, D-Val, D-Trp, and D-Tyr.

In certain embodiments, Xₐₐ⁸ is selected from D-Met, D-Leu, and D-Thr.

In certain embodiments, Xₐₐ⁹ is an unsubstituted D-amino acid residue selected from D-Phe, D-His, D-Leu, D-Pro, D-Ser, D-Glu, D-Thr, D-Val, D-Trp, and D-Tyr.

In certain embodiments, Xₐₐ⁹ is selected from D-Ser, D-Thr, and D-Tyr.

In certain embodiments, Xₐₐ¹⁰ is an unsubstituted D-Lys.

In certain embodiments, Xₐₐ¹⁰ is substituted D-Lys.

In certain embodiments, the conjugate having the structure:

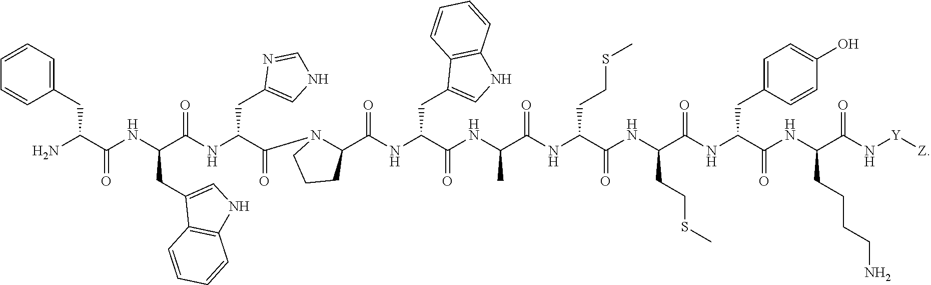

In certain embodiments, the conjugate having the structure:

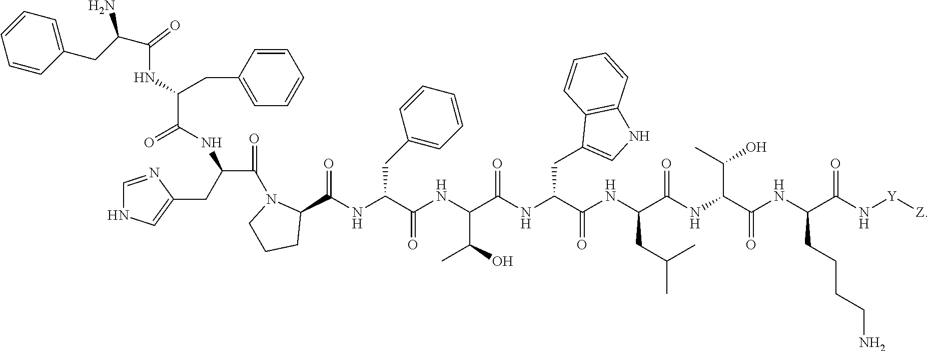

In certain embodiments, the conjugate having the structure:

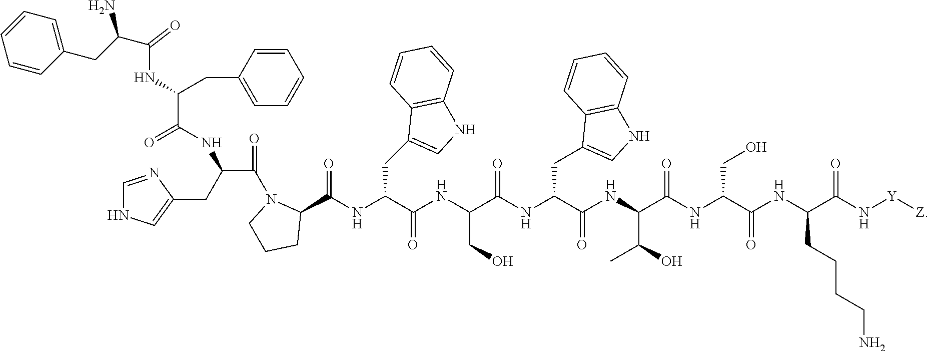

In certain embodiments, the conjugate having the structure selected from:

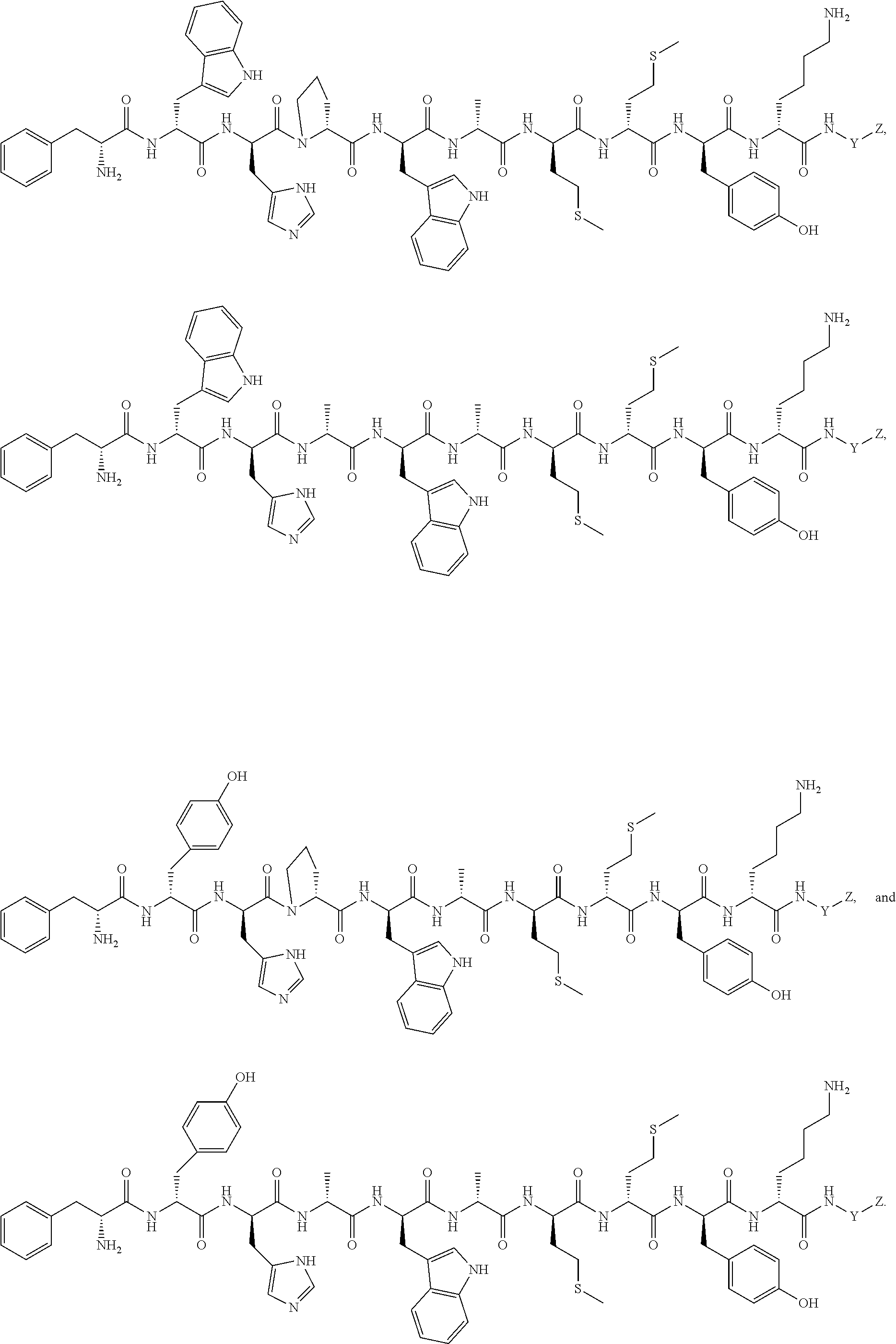

In certain embodiments, Y has the structure -L₁-, -L₁-L₂-, or -L₁-S₁-L₂-

wherein

L₁ is a first linker unit;

S₁ is a spacer; and
L₂ is a second linker unit. In certain embodiments, wherein Y has the structure -L₁-S₁-L₂-,

wherein

L₁ is a first linker unit;

S₁ is an optional spacer; and

L₂ is a second linker unit; wherein L₂ is bonded to the active moiety.

In certain embodiments, L₁ is a peptide.

In certain embodiments, L₁ comprises 4 to 20 amino acid residues.

In certain embodiments, L₁ comprises at least one Gly.

In certain embodiments, L₁ further comprises at least one optionally substituted D-Lys or D-Cys.

In certain embodiments, L₁ comprises at least one internal D-Lys or D-Cys.

In certain embodiments, L₁ comprises at least one internal substituted D-Lys or D-Cys.

In certain embodiments, L₁ comprises at least one internal L-Lys or L-Cys.

In certain embodiments, L₁ comprises at least one internal substituted L-Lys or L-Cys.

In certain embodiments, the sidechain amino group of the internal D-Lys is substituted with

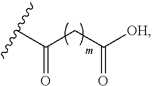

wherein m is 4-20.

In certain embodiments, the sidechain amino group of the internal D-Lys or the sidechain thio group of the internal D-Cys is substituted with a second peptide of the invention.

In certain embodiments, the sidechain amino group of the internal L-Lys or the sidechain thio group of the internal L-Cys is substituted with a second peptide of the invention.

In certain embodiments, the sidechain amino group of the internal D-Lys or the sidechain thio group of the internal D-Cys is substituted with a second peptide.

In certain embodiments, the second peptide has the structure:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-
Xₐₐ¹⁰-R_{C}-L₁′-

[symbol]

wherein L₁′ is a third linker unit.

In certain embodiments, L₁′ is a peptide.

In certain embodiments, L₁′ comprises 4 to 20 amino acid residues.

In certain embodiments, L₁′ comprises at least one Gly.

In certain embodiments, L₁′ further comprises at least one optionally substituted D-Lys or D-Cys.

In certain embodiments, L₁′ further comprises at least one optionally substituted L-Lys or L-Cys.

In certain embodiments, L₁′ terminates at the C-terminus with a Gly, D-Lys, or D-Cys which is bonded to the internal D-Lys or internal D-Cys of L₁.

In certain embodiments, L₁′ terminates at the C-terminus with an L-Lys, or L-Cys which is bonded to the internal D-Lys or internal D-Cys of L₁.

In certain embodiments, L₁′ terminates at the C-terminus with a Gly, D-Lys, or D-Cys which is bonded to the internal L-Lys or internal L-Cys of L₁.

In certain embodiments, L₁′ terminates at the C-terminus with an L-Lys, or L-Cys which is bonded to the internal L-Lys or internal L-Cys of L₁.

In certain embodiments, the sidechain amino group of the C-terminal D-Lys of L₁′ or the sidechain thio group of the C-terminal D-Cys of L₁′ is substituted with a third peptide of the invention.

In certain embodiments, the sidechain amino group of the C-terminal L-Lys of L₁′ or the sidechain thio group of the C-terminal L-Cys of L₁′ is substituted with a third peptide of the invention.

In certain embodiments, the sidechain amino group of the C-terminal D-Lys of L₁′ is substituted with an additional peptide or the sidechain thio group of the C-terminal D-Cys of L₁′ is substituted with a third peptide.

In certain embodiments, the additional peptide has the structure:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-
Xₐₐ¹⁰-R_{C}-L₁″-

[symbol]

wherein L₁″ is a fourth linker unit.

In certain embodiments, L₁″ is a peptide.

In certain embodiments, L₁″ comprises 4 to 20 amino acid residues.

In certain embodiments, L₁″ comprises at least one Gly.

In certain embodiments, each amino acid residue is Gly.

In certain embodiments, L₁″ further comprises at least one optionally substituted D-Lys or D-Cys.

In certain embodiments, L₁″ further comprises at least one optionally substituted L-Lys or L-Cys.

In certain embodiments, L₁ terminates at the C-terminus with a D-Lys or D-Cys.

In certain embodiments, S₁ is present; and the sidechain amino group of the terminal D-Lys of L₁ is bonded to S₁.

In certain embodiments, S₁ is present; and the thio group of the terminal D-Cys is bonded to S₁.

In certain embodiments, S₁ comprises at least two keto (oxo) groups.

In certain embodiments, S₁ has the structure:

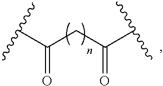

wherein n is 2 to 20.

In certain embodiments, the sidechain amino group of the terminal D-Lys of L₁ is bonded to L₂.

In certain embodiments, the sidechain thio group of the terminal D-Cys of L₁ is bonded to L₂.

In certain embodiments, L₂ comprises a thiosuccinimide moiety.

In certain embodiments, L₂ comprises a 1,2,3-triazole moiety.

In certain embodiments, Y is -L₁-. In other embodiments, Y is -L₁-S₁-L₂-. In other embodiments, L₁ is a peptide.

In certain embodiments, L₁ comprises 2 to 20 amino acid residues.

In certain embodiments, L₁ comprises at least one Gly.

In certain embodiments, L₁ further comprises at least one internal optionally substituted L-Lys or L-Cys.

In certain embodiments, L₁ comprises at least one internal substituted L-Lys or L-Cys.

In certain embodiments, the sidechain amino group of the internal L-Lys or the sidechain thio group of the internal L-Cys is substituted with a peptide.

In certain embodiments, the second peptide has the structure:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-
Xₐₐ¹⁰-R_{C}-L₁′-

[symbol]

wherein L₁′ is a third linker unit.

In certain embodiments, L₁′ is a peptide.

In certain embodiments, L₁′ comprises 2 to 20 amino acid residues.

In certain embodiments, L₁′ comprises at least one Gly.

In certain embodiments, L₁ terminates at the C-terminus with an L-Lys or L-Cys.

In certain embodiments, S₁ is present; and the sidechain amino group of the terminal L-Lys of L₁ is bonded to S₁.

In certain embodiments, S₁ is present; and the thio group of the terminal L-Cys of L₁ is bonded to S₁.

In certain embodiments, S₁ comprises at least one keto (oxo) group.

In certain embodiments, S₁ has the structure:

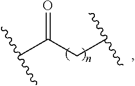

wherein n is 2 to 20.

In certain embodiments, S₁ has the structure:

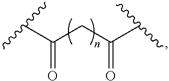

wherein n is 2 to 20.

In certain embodiments, L₂ comprises a succinimide moiety.

In certain embodiments, L₂ comprises a 1,2,3-triazole moiety.

In certain embodiments, L₂ comprises a PEG moiety.

In certain embodiments, L₁ comprises a PEG moiety.

In certain embodiments, L₁ comprises two PEG moieties.

In certain embodiments, L₁ terminates at the C-terminus with a L-Lys, L-Cys, D-Lys or D-Cys.

In certain embodiments, L₁ has the structure

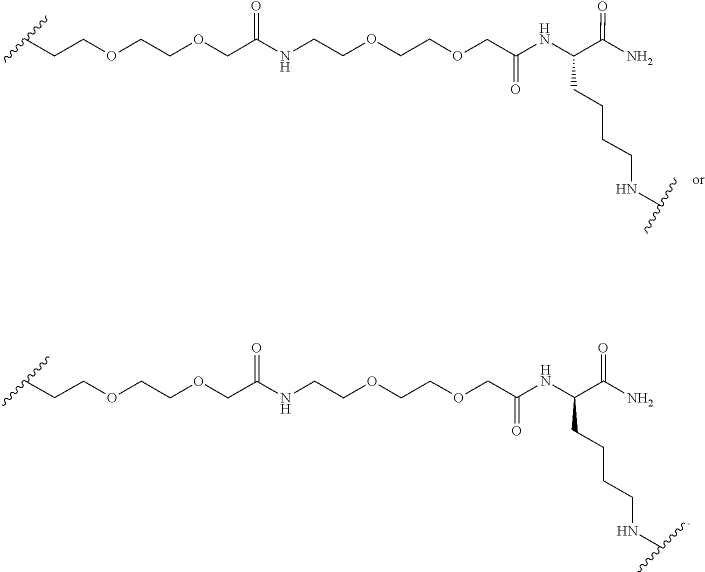 [SMILES: *CCOCCOCC(=O)NCCOCCOCC(=O)N[C@@H](CCCCN*)C(N)=O ; *CCOCCOCC(=O)NCCOCCOCC(=O)N[C@H](CCCCN*)C(N)=O]

In certain embodiments, L₁ comprises at least one Gly.

In certain embodiments, L₁ comprises four Gly.

In certain embodiments, L₁ terminates at the C-terminus with a L-Lys, L-Cys, D-Lys or D-Cys.

In certain embodiments, L₁ has the structure

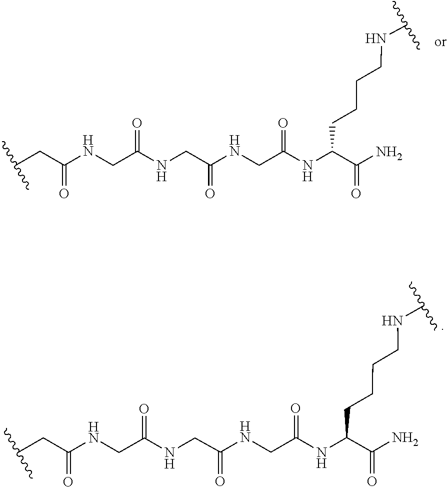 [SMILES: *CC(=O)NCC(=O)NCC(=O)NCC(=O)N[C@H](CCCCN*)C(N)=O ; *CC(=O)NCC(=O)NCC(=O)NCC(=O)N[C@@H](CCCCN*)C(N)=O]

In certain embodiments, Z is a drug moiety. In other embodiments, Z is a non-immunostimulant.

In certain embodiments, Z is a biologic. In other embodiments, Z is a therapeutic antibody. In other embodiments, Z is a non-biologic. In other embodiments, Z is a small molecule. In other embodiments, Z is a peptide. In certain embodiments, Z is a natural product.

In certain embodiments, the therapeutic antibody is a monoclonal antibody. In certain embodiments, the therapeutic antibody is Adalimumab.

In certain embodiments, Z is a GLP-1 agonist, e.g., a peptide or a GLP-1 peptide variant.

In certain embodiments, Z is a dulaglutide, exenatide, semaglutide, liraglutide, and lixisenatide.

In certain embodiments, Z is an erythrocyte-targeted antigen moiety.

In certain embodiments, Z is selected from an antibody, virus, hormone, enzyme, peptide, protein, and single-chain variable fragment (scFv).

In certain embodiments, Z is selected from immunoglobulin G (IgG), adeno-associated virus (AAV), and human growth hormone (HGH).

In certain embodiments, Z is insulin.

In certain embodiments, Z is a biotin moiety. In certain embodiments, the conjugate has the structure:

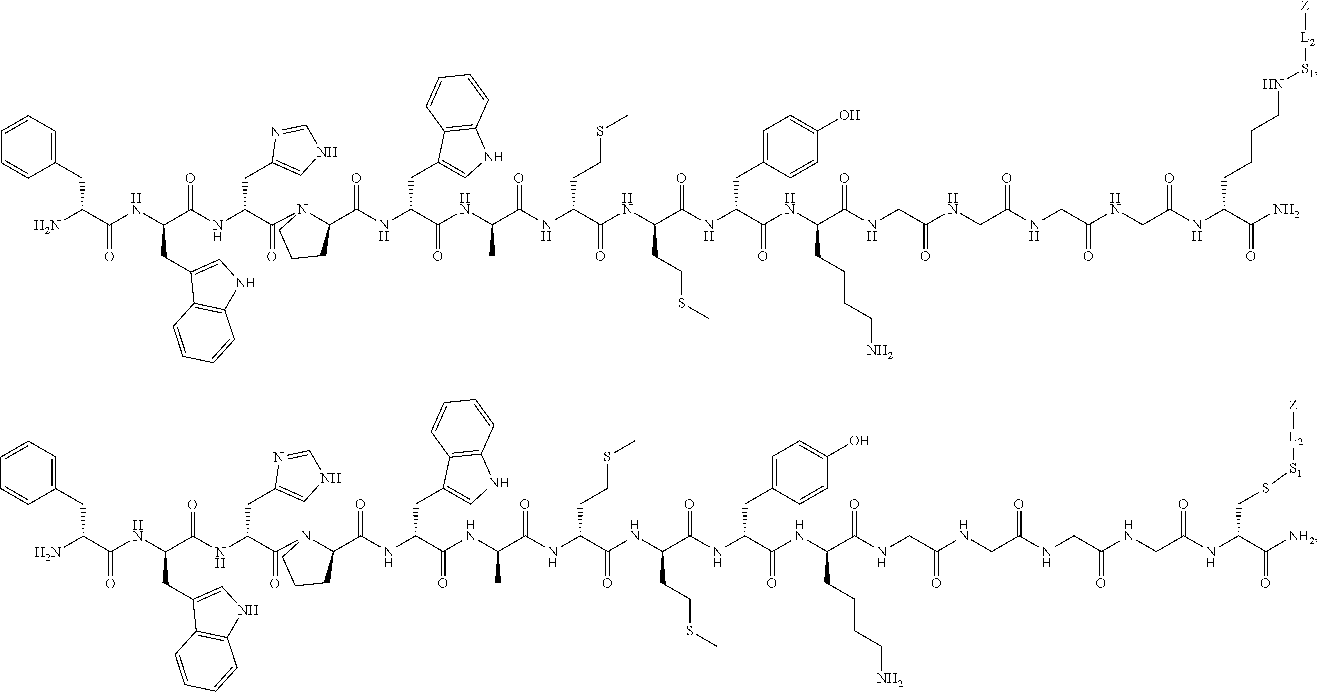

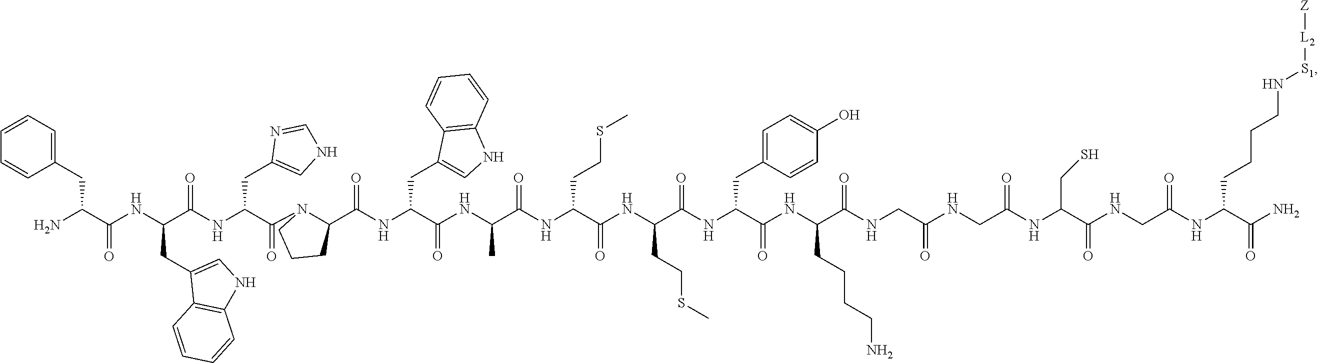

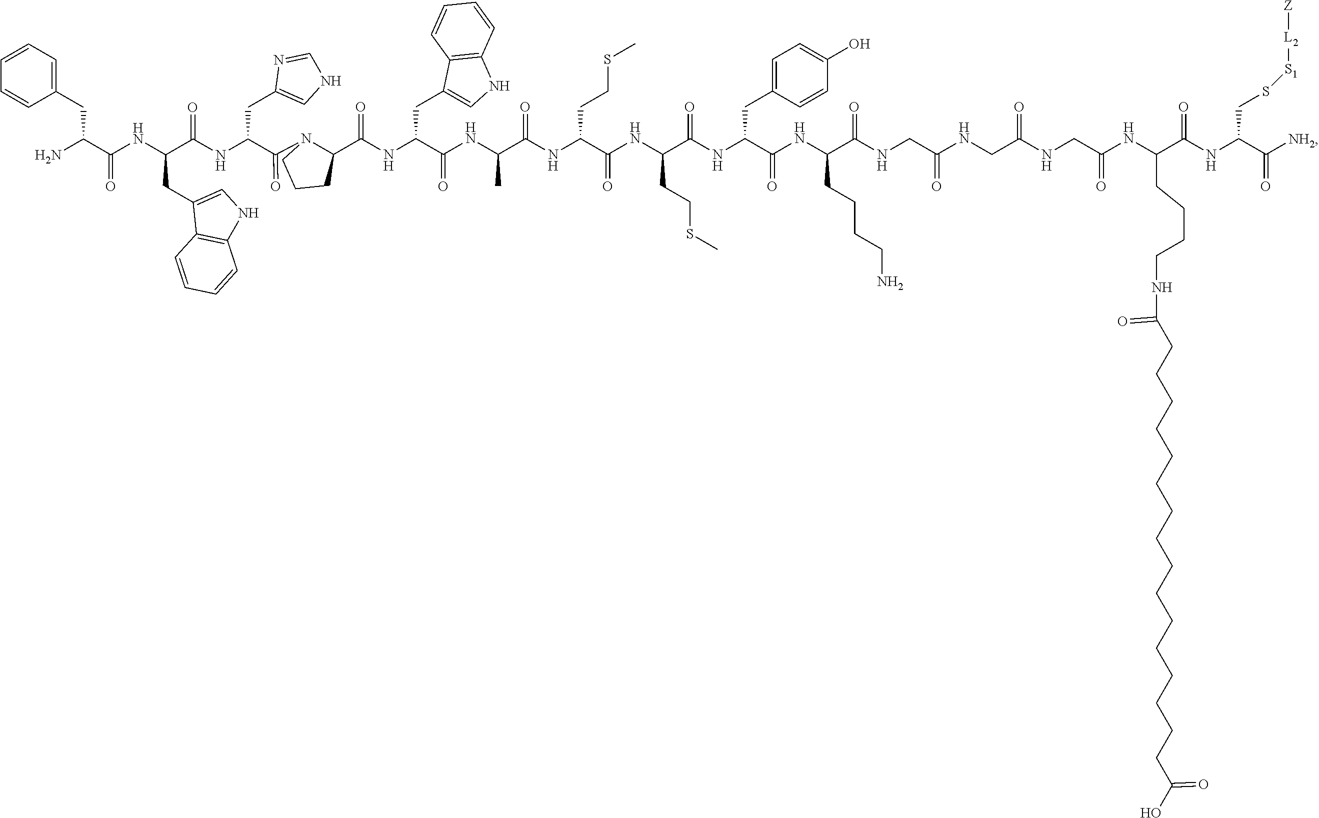

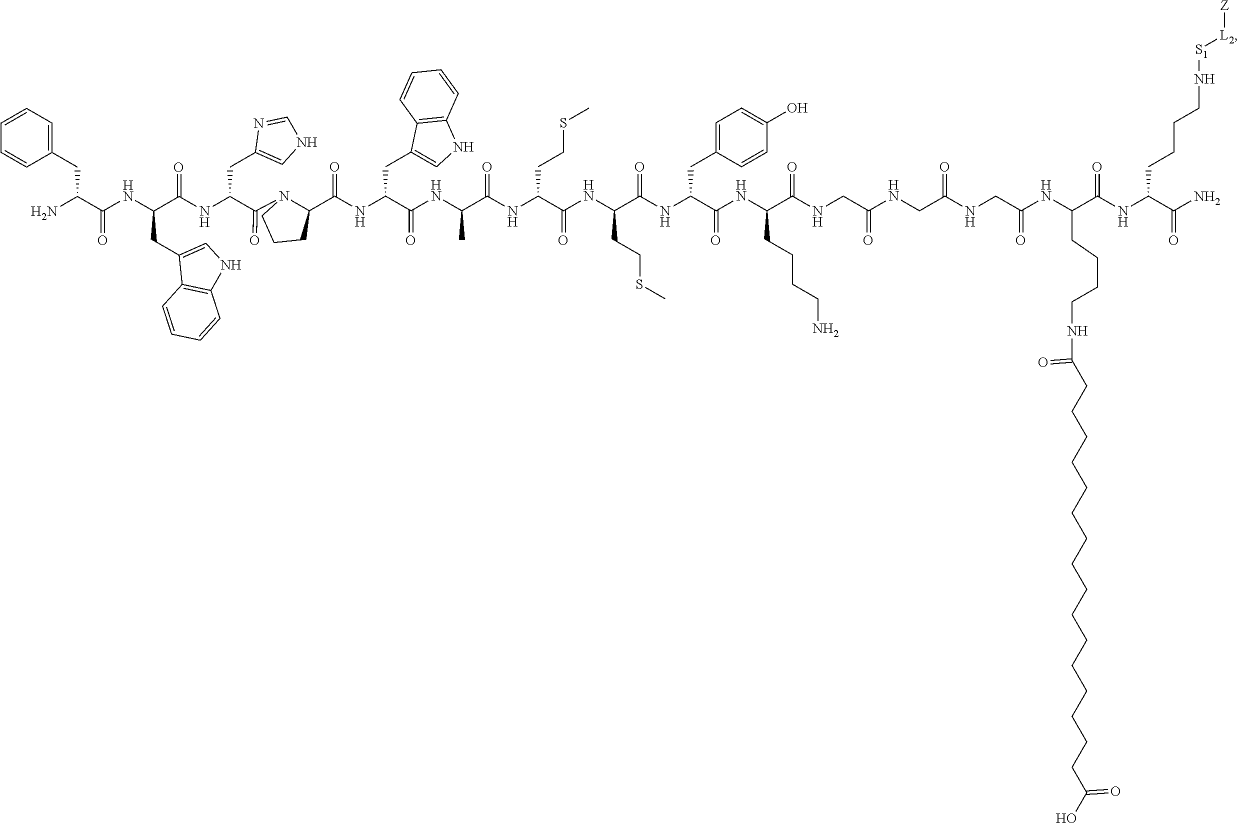

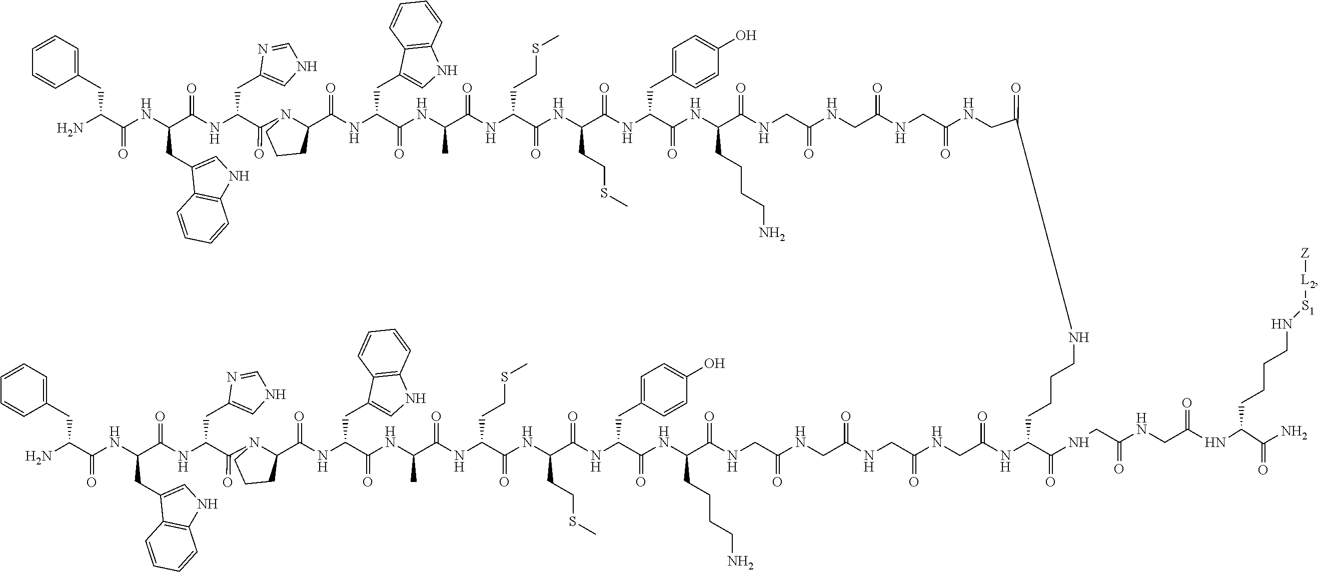

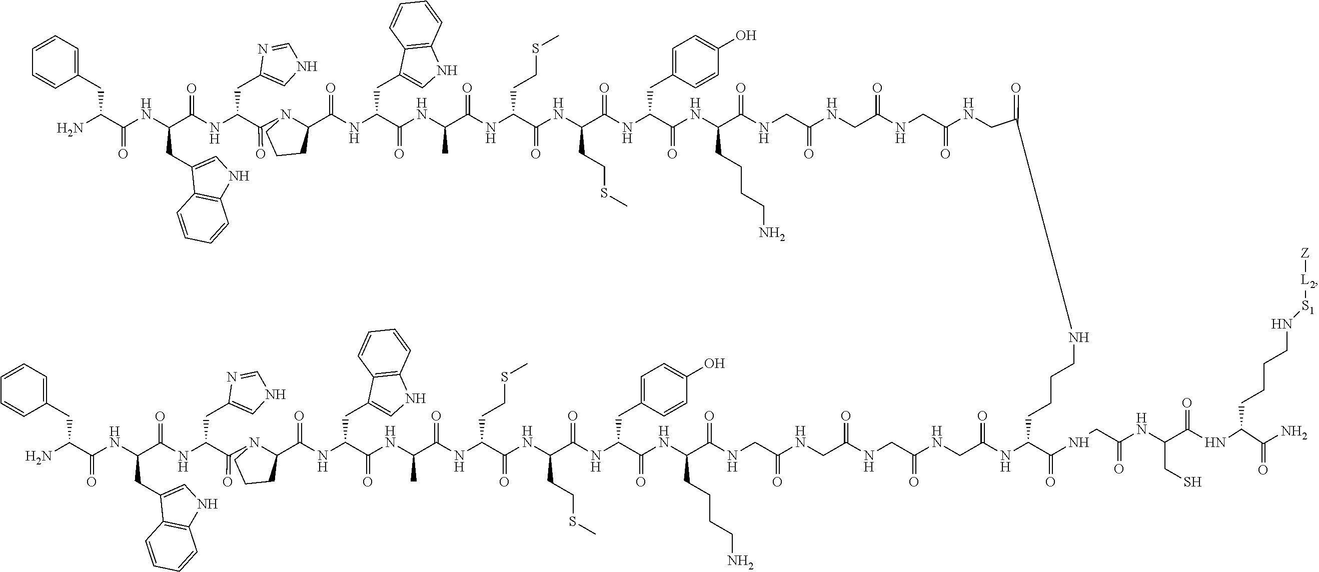

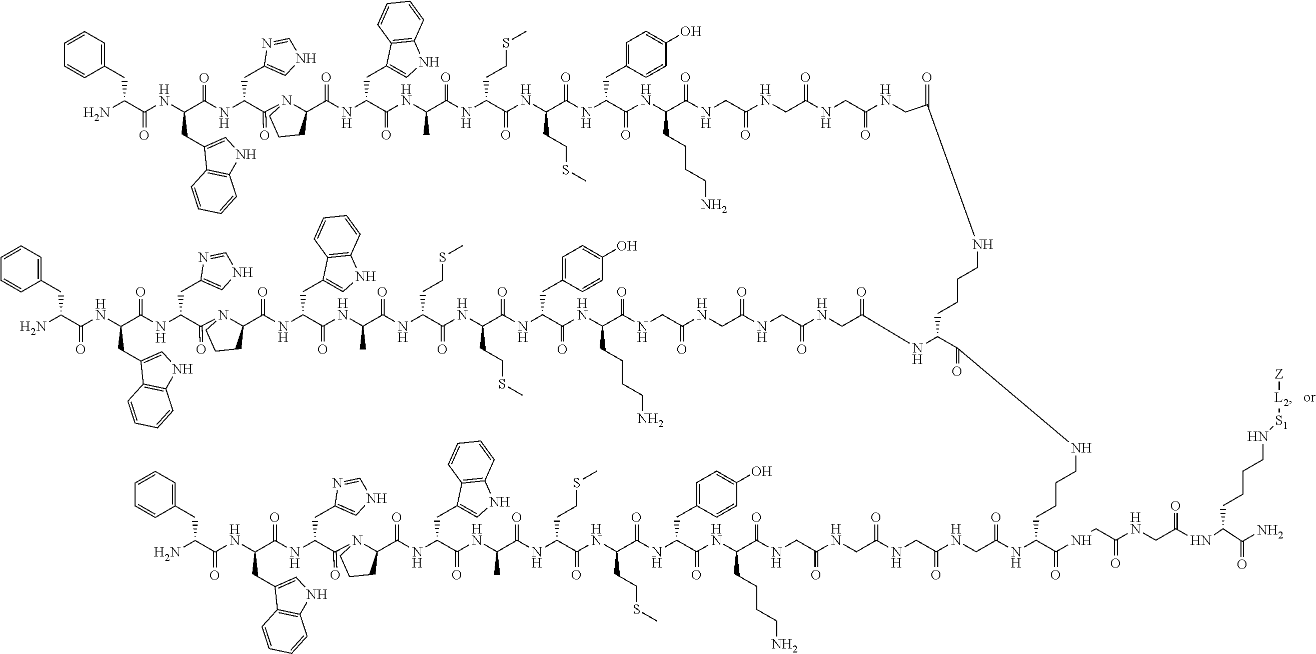

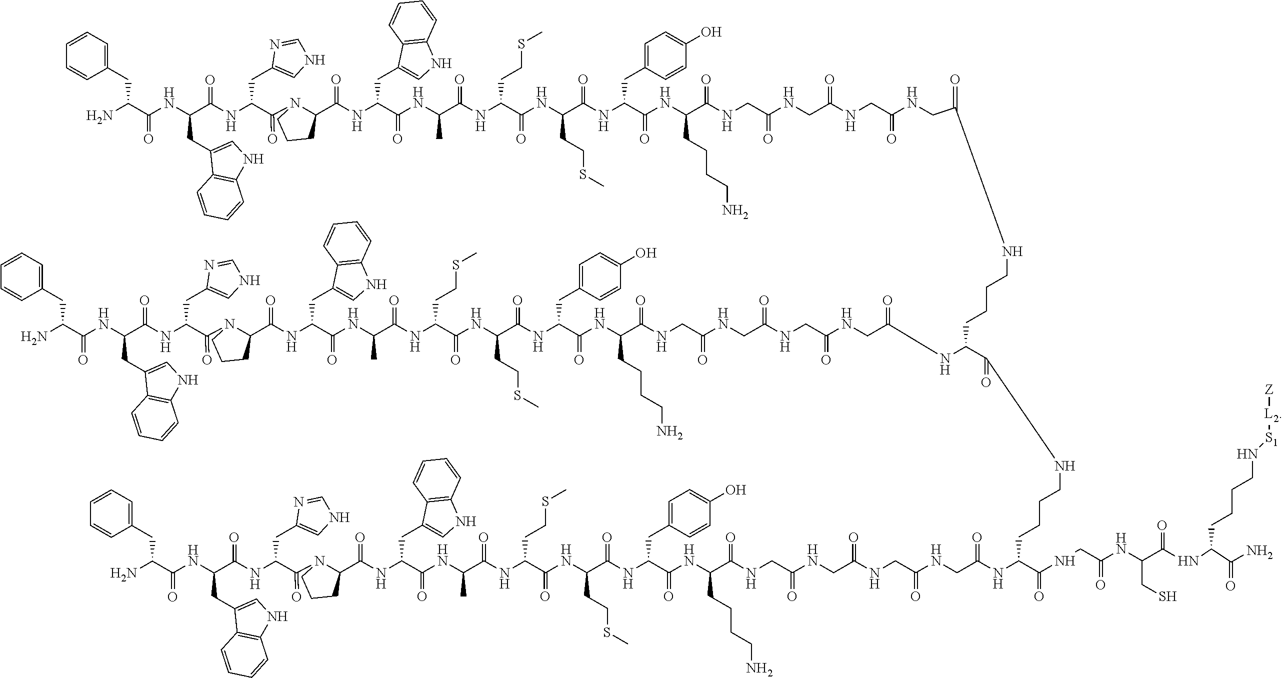

In certain embodiments, the conjugate has the structure:

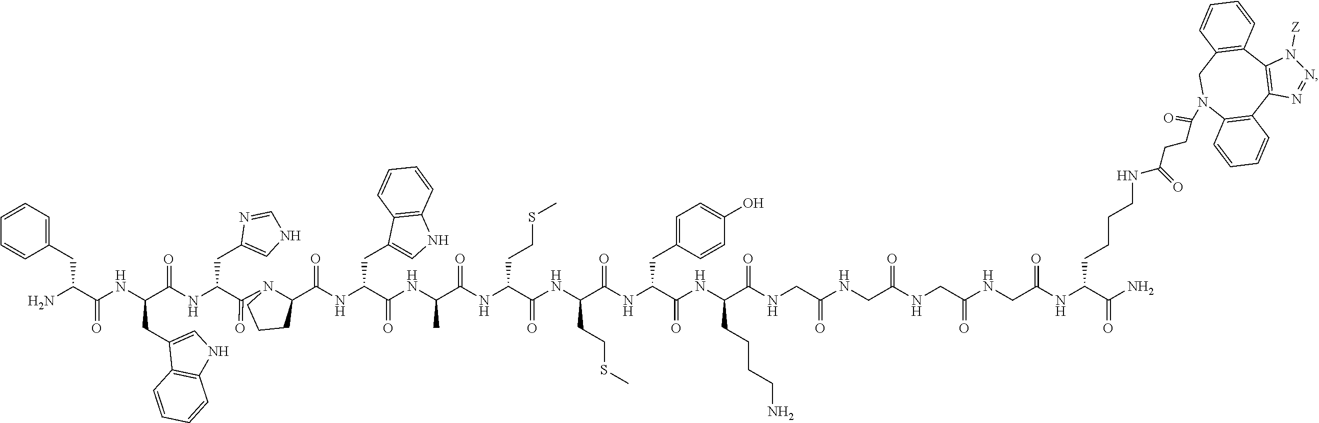

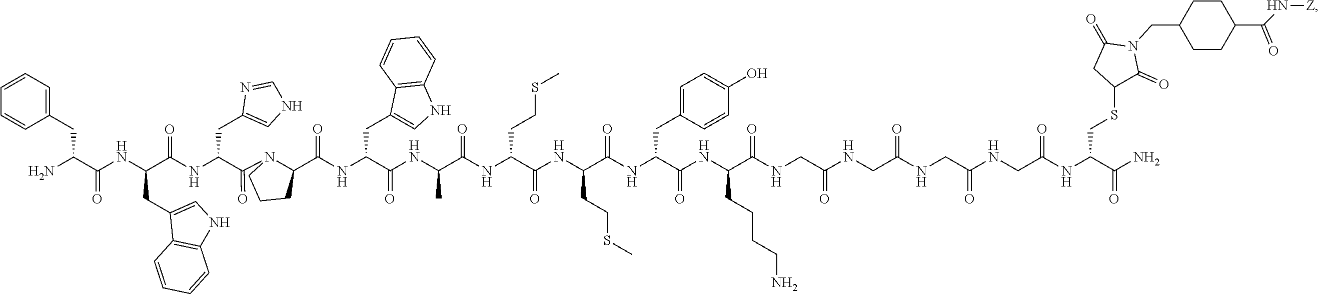

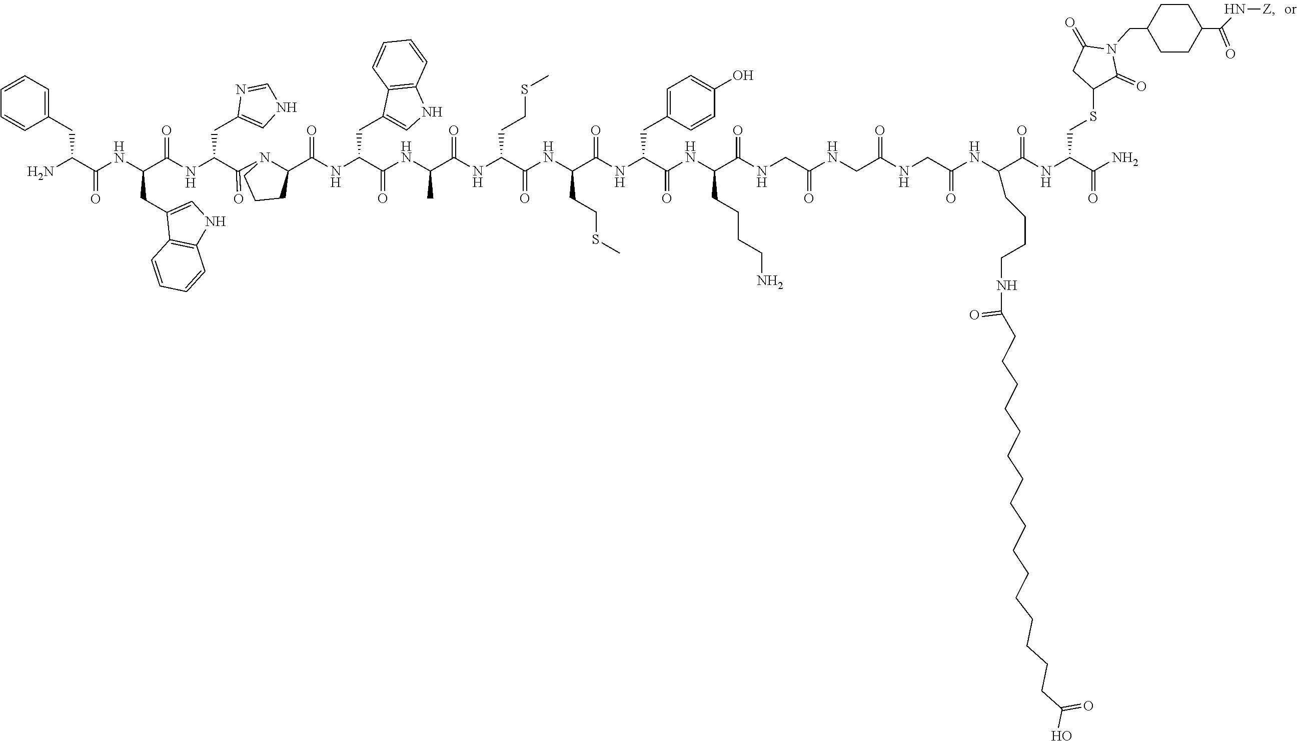

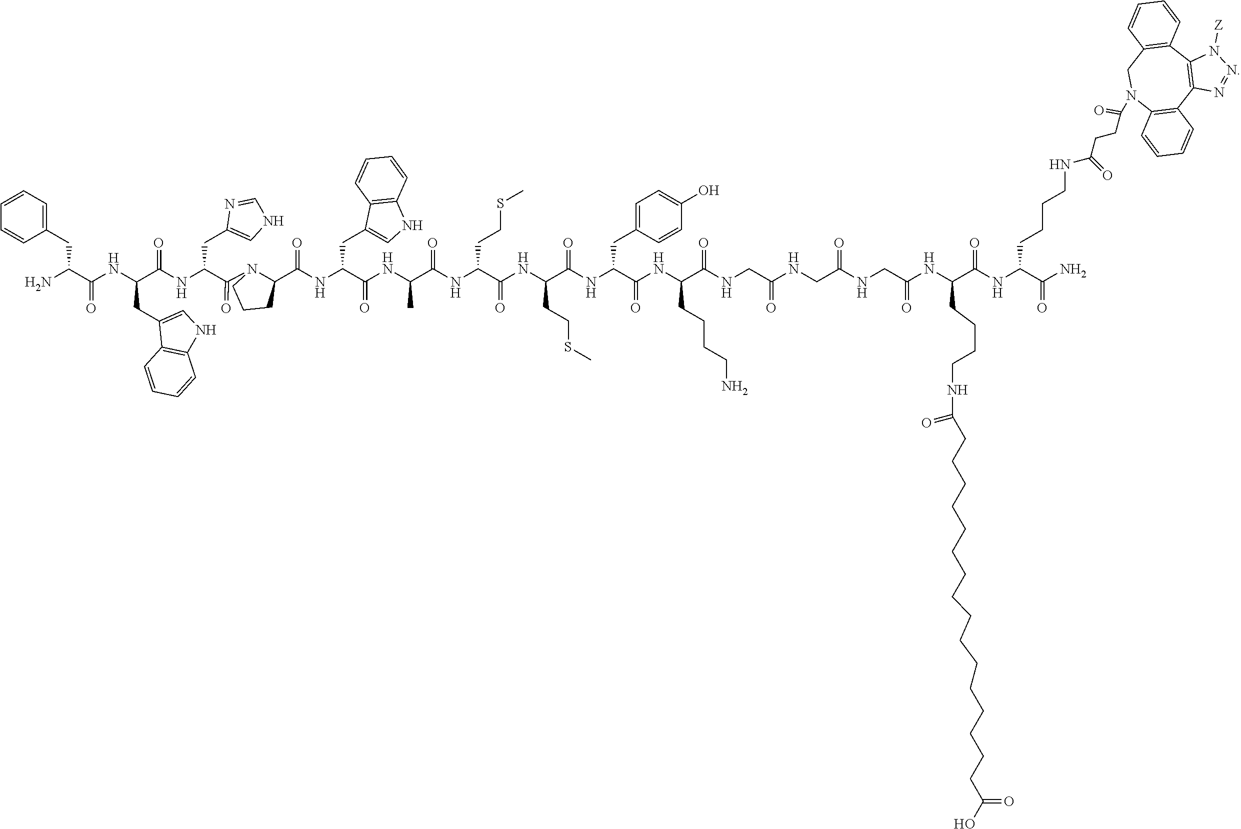

In certain embodiments, the conjugate has the structure:

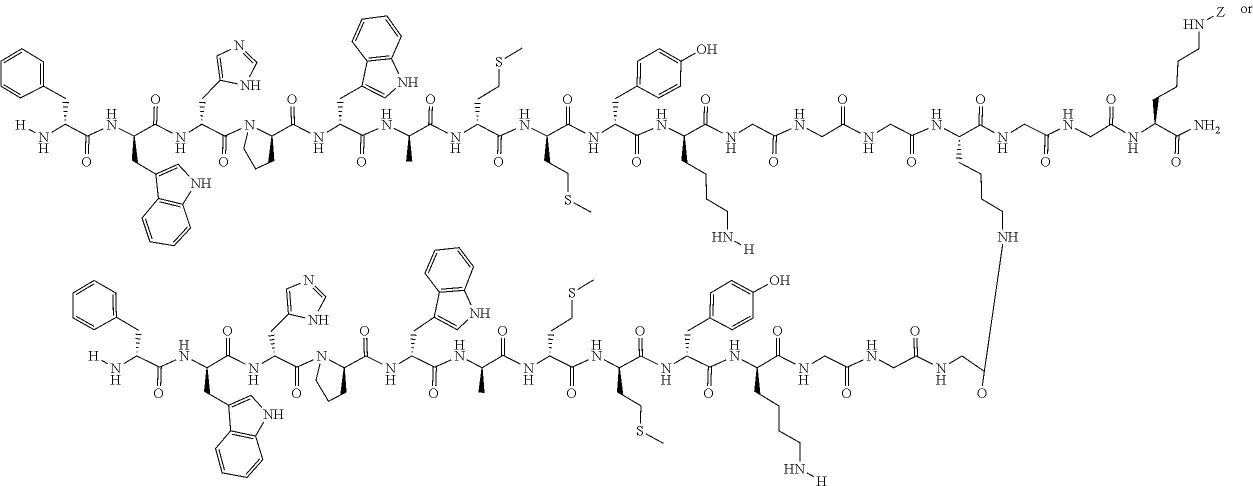

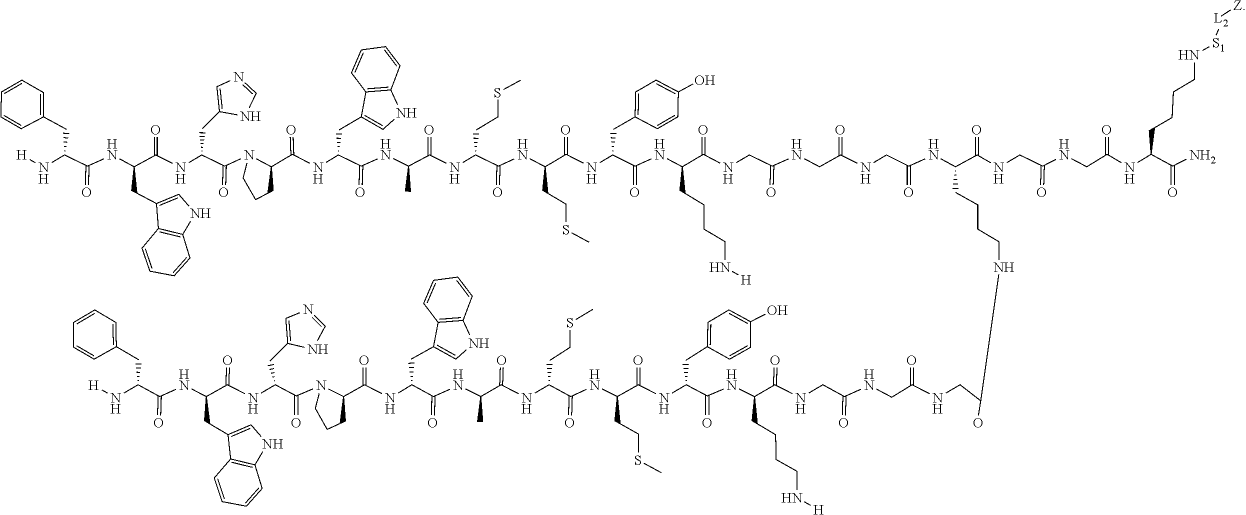

In certain embodiments, the conjugate has the structure:

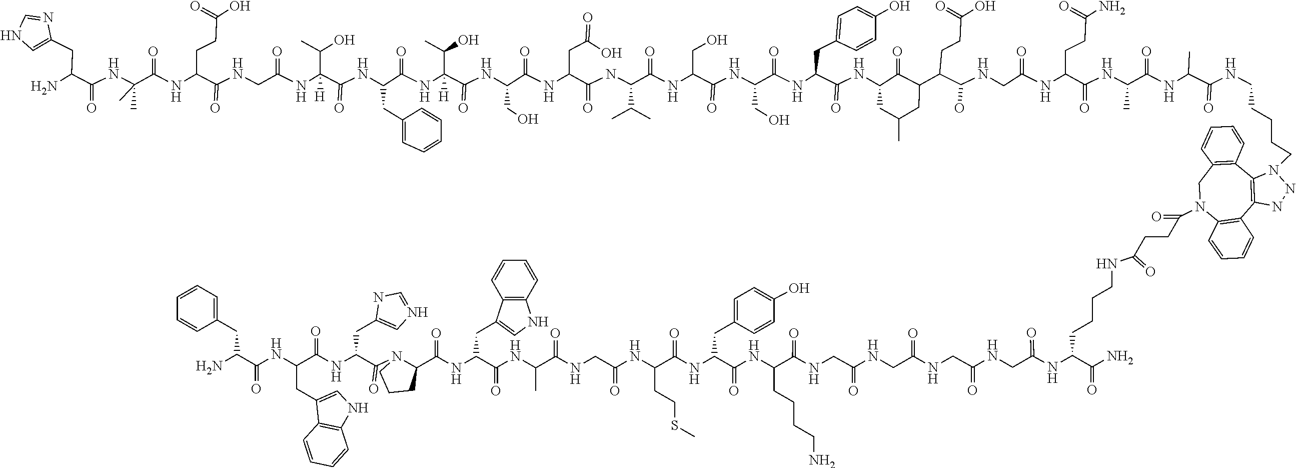

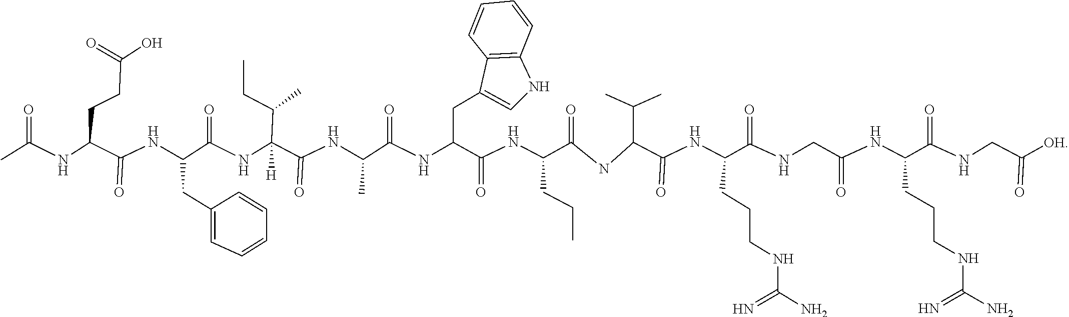

In certain embodiments, the conjugate has the structure:

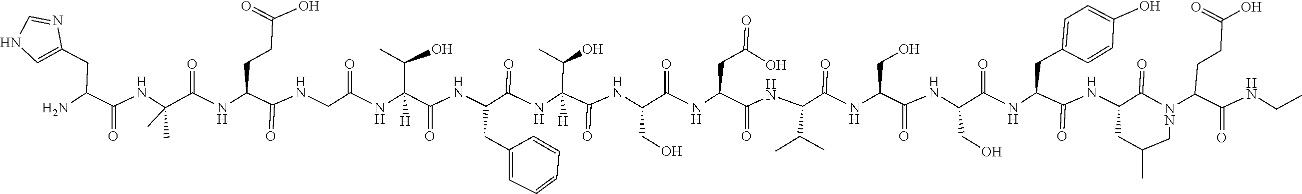

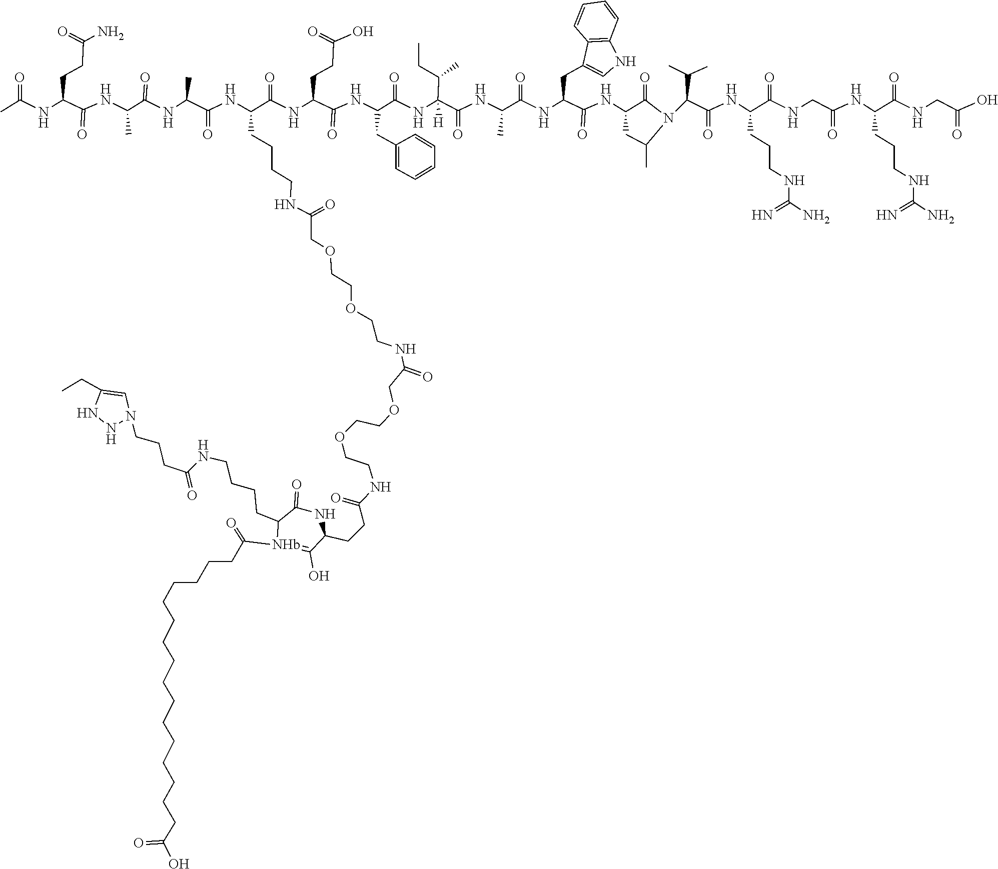

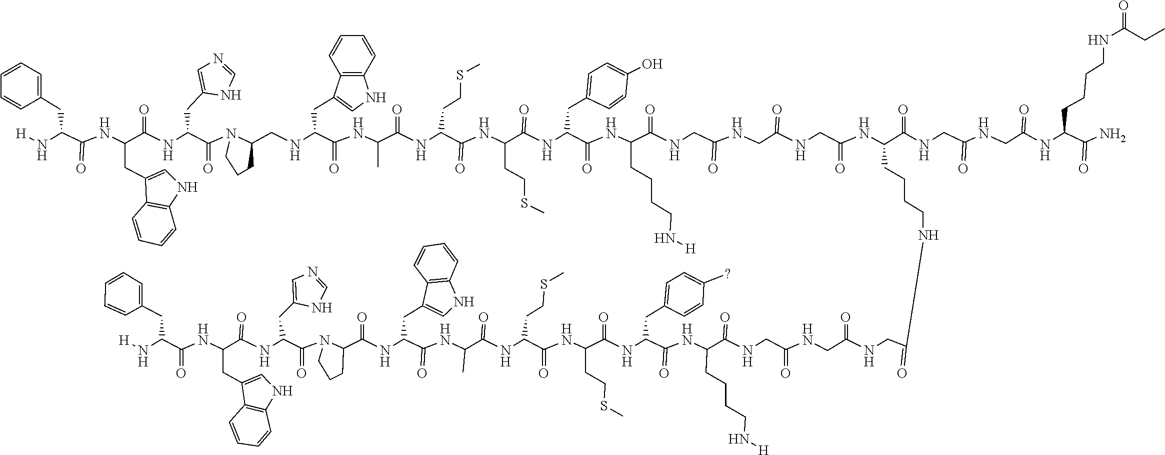

In certain embodiments, the conjugate has the structure:

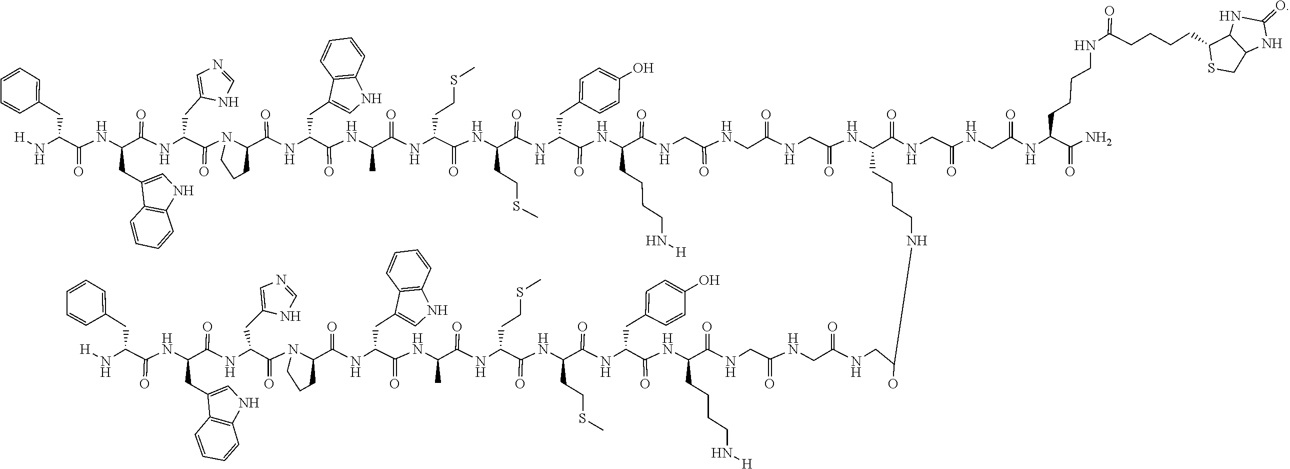

In certain embodiments, the conjugate has the structure:

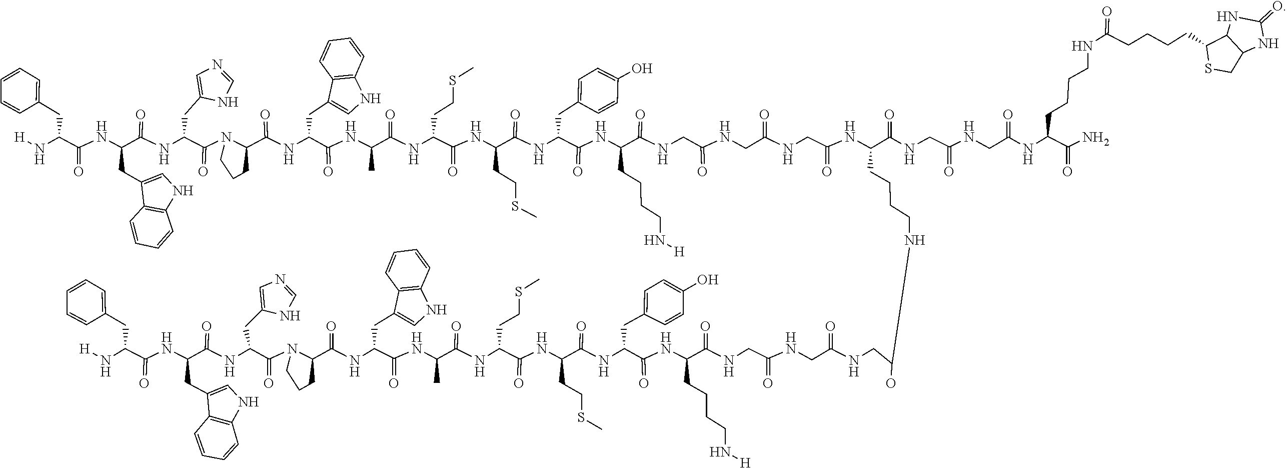

In certain embodiments, the conjugate has the structure:

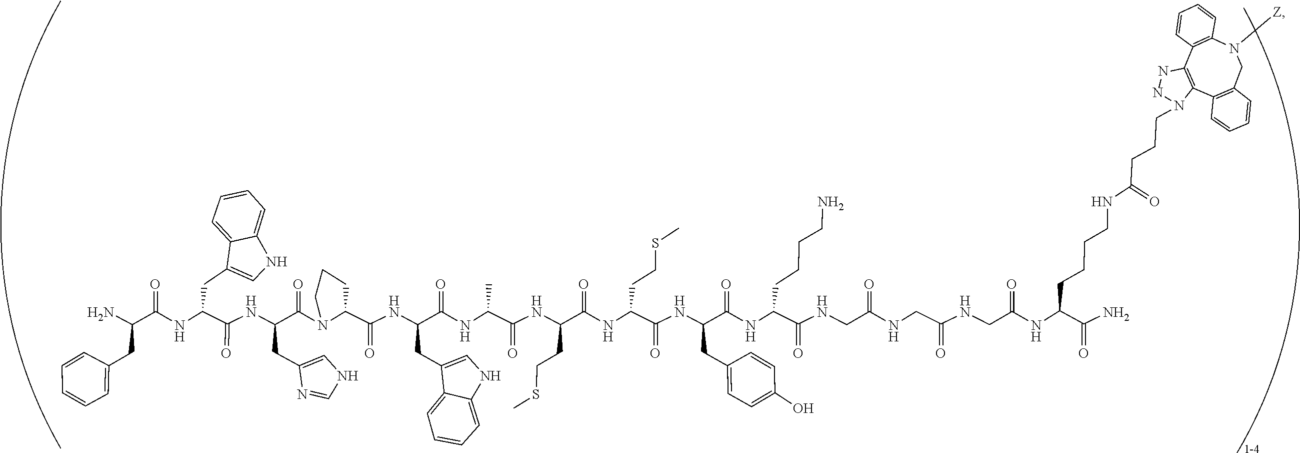

wherein Z is a therapeutic antibody.

In certain embodiments, the conjugate has the structure:

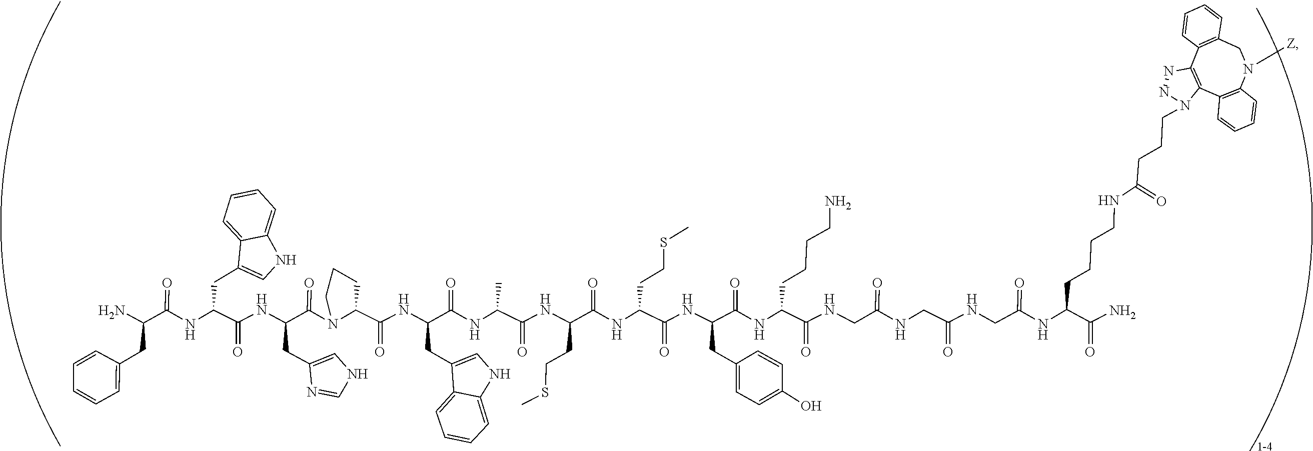

wherein Z is a therapeutic antibody.

In certain embodiments, the therapeutic antibody is Adalimumab.

In certain embodiments, the amino acid residue is unsubstituted. In other embodiments, the amino acid residue is substituted, e.g., with at least one halo, hydroxyl, alkyl, or alkynyl. In certain embodiments, the thiol group of Cys is alkylated.

In certain embodiments, the D-amino acid residue is unsubstituted. In other embodiments, the D-amino acid residue is substituted, e.g., with at least one halo, hydroxyl, alkyl, or alkynyl. In certain embodiments, the thiol group of Cys is alkylated.

In certain embodiments, one or more of Xₐₐ⁴, Xₐₐ⁵, Xₐₐ⁶ and Xₐₐ⁹ is substituted D-Pro, wherein the subsitutent is halo, hydroxyl, alkyl, or alkynyl.

In certain embodiments, one or more of Xₐₐ⁴, Xₐₐ⁵, Xₐₐ⁶ and Xₐₐ⁹ is a D-Pro derivative residue.

In certain embodiments, one or more of Xₐₐ⁴, Xₐₐ⁵, Xₐₐ⁶ and Xₐₐ⁹ is a cyclic D-amino acid residue.

Also provided herein is a conjugate, comprising a peptide moiety, a linker, and an active moiety, having the following structure of formula (I):

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)

wherein

R_{N} is the terminal H₂N— group of Xₐₐ¹;

Xₐₐ¹ is an optionally substituted D-amino acid residue selected from D-Phe and D-Leu;

Xₐₐ² is an optionally substituted D-amino acid residue selected from D-Phe, D-Trp, D-Tyr, and D-Ala;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Phe, D-Ala, D-His, D-Lys, D-Met, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-amino acid residue selected from D-Ala, D-Lys, and D-His;

R_{C} is the C-terminal —C(O)—NH— group of Xₐₐ¹⁰;

Y is the linker which links the peptide moiety and the active moiety; and

Z is the active moiety.

Also provided herein is a conjugate, wherein each of amino acids 1 to 10 are in the D-configuration except for each occurrence of Gly, NMeGly and L-Pro, selected from:

| | |
|---|---|
| 1a | H-Phe-Trp-His-Pro-Trp-Ala-Met-Met-Tyr-Lys-Gly-Gly-Gly-Lys(Biotin)-NH₂ |
| 1b | H-Ala-Trp-His-Pro-Trp-Ala-Met-Met-Tyr-Lys-Gly-Gly-Gly-Lys(Biotin)-NH₂ |
| 1c | H-Phe-Ala-His-Pro-Trp-Ala-Met-Met-Tyr-Lys-Gly-Gly-Gly-Lys(Biotin)-NH₂ |
| 1d | H-Phe-Trp-Ala-Pro-Trp-Ala-Met-Met-Tyr-Lys-Gly-Gly-Gly-Lys(Biotin)-NH₂ |
| 1e | H-Phe-Trp-His-Ala-Trp-Ala-Met-Met-Tyr-Lys-Gly-Gly-Gly-Lys(Biotin)-NH₂ |
| 1f | H-Phe-Trp-His-Pro-Ala-Ala-Met-Met-Tyr-Lys-Gly-Gly-Gly-Lys(Biotin)-NH₂ |
| 1g | H-Phe-Trp-His-Pro-Trp-Ala-Ala-Met-Tyr-Lys-Gly-Gly-Gly-Lys(Biotin)-NH₂ |
| 1h | H-Phe-Trp-His-Pro-Trp-Ala-Met-Ala-Tyr-Lys-Gly-Gly-Gly-Lys(Biotin)-NH₂ |
| 1i | H-Phe-Trp-His-Pro-Trp-Ala-Met-Met-Ala-Lys-Gly-Gly-Gly-Lys(Biotin)-NH₂ |
| 1j | H-Phe-Trp-His-Pro-Trp-Ala-Met-Met-Tyr-Ala-Gly-Gly-Gly-Lys(Biotin)-NH₂ |
| 2a | H-Phe-Trp-His-Pro-Trp-Ala-Nle-Nle-Tyr-Lys-Gly-Gly-Gly-Lys(Biotin)-NH₂ |
| 2b | H-Phe-Trp-His-Pro-Trp-Ala-Ala-Ala-Tyr-Lys-Gly-Gly-Gly-Lys(Biotin)-NH₂ |
| 2c | H-Phe-Trp-His-Pro-Trp-Ala-Leu-Leu-Tyr-Lys-Gly-Gly-Gly-Lys(Biotin)-NH₂ |
| 2d | H-Phe-Trp-His-Pro-Trp-Ala-Met-Met-Tyr-Lys-Gly-Gly-Gly-(H-Phe-Trp-His-Pro-Trp-Ala- |
| | Met-Met-Tyr-Lys-Gly-Gly-Gly)-Lys-Gly-Gly-Lys(Biotin)-NH₂ |
| 2e | H-Phe-Trp-His-Pro-Trp-Ala-Met-Met-Tyr-Lys-Gly-Gly-Gly-Lys(C18diacid)-Lys(Biotin)-NH₂ |
| 2f | H-Ala-Pro-Met-Phe-Tyr-Trp-Lys-His-Met-Trp-Gly-Gly-Gly-Lys(Biotin)-NH₂ |
| 2g | Ac-Phe-Trp-His-Pro-Trp-Ala-Met-Met-Tyr-Lys-Gly-Gly-Gly-Lys(Biotin)-NH₂ |
| 2h | H-Phe-Trp-His-Pro-Trp-Ala-Met-Met-Tyr-Lys-Gly-Gly-Gly-Cys(Trt)-Gly-Lys(Biotin)-NH₂ |
| 2i | H-Phe-Trp-Ser-Pro-Trp-Ala-Met-Met-Tyr-Lys-Gly-Gly-Gly-Lys(Biotin)-NH₂ |
| 2j | H-Phe-Trp-Tyr-Pro-Trp-Ala-Met-Met-Tyr-Lys-Gly-Gly-Gly-Lys(Biotin)-NH₂ |
| 2k | H-Phe-Trp-His-N-MeGly-Trp-Ala-Met-Met-Tyr-Lys-Gly-Gly-Gly-Lys(Biotin)-NH₂ |
| 2l | H-Phe-Trp-His-L-Pro-Trp-Ala-Met-Met-Tyr-Lys-Gly-Gly-Gly-Lys(Biotin)-NH₂ |
| 2m | H-Phe-Trp-His-αMePro-Trp-Ala-Met-Met-Tyr-Lys-Gly-Gly-Gly-Lys(Biotin)-NH₂ |
| 3a | H-Phe-Phe-His-Pro-Trp-Ser-Trp-Thr-Ser-Lys-Gly-Gly-Gly-Lys(Biotin)-NH₂ |
| 3b | H-Phe-Phe-His-Pro-Tyr-Gly-Leu-Phe-Val-Lys-Gly-Gly-Gly-Lys(Biotin)-NH₂ |
| 3c | H-Phe-Trp-His-Pro-Trp-Phe-Phe-Lys-Thr-Lys-Gly-Gly-Gly-Lys(Biotin)-NH₂ |
| 3d | H-Phe-Trp-Val-Pro-Phe-Trp-Trp-Lys-Thr-Lys-Gly-Gly-Gly-Lys(Biotin)-NH₂ |
| 3e | H-Leu-His-His-Pro-Trp-Pro-Tyr-Phe-Tyr-Lys-Gly-Gly-Gly-Lys(Biotin)-NH₂ |
| 3f | H-Leu-Trp-His-Pro-Tyr-Leu-Phe-Leu-Trp-Lys-Gly-Gly-Gly-Lys(Biotin)-NH₂ |
| 3g | H-Ser-Trp-His-Pro-Val-Trp-Gly-Trp-Leu-Lys-Gly-Gly-Gly-Lys(Biotin)-NH₂ |
| 3h | H-Phe-Phe-His-Pro-Phe-Ala-Trp-Phe-His-Lys-Gly-Gly-Gly-Lys(Biotin)-NH₂ |
| 3i | H-Phe-Phe-His-Val-Pro-Val-Phe-Ser-Val-Lys-Gly-Gly-Gly-Lys(Biotin)-NH₂ |
| 3j | H-Phe-Trp-Tyr-Val-His-Thr-Thr-Gly-Trp-Lys-Gly-Gly-Gly-Lys(Biotin)-NH₂ |
| 3k | H-Phe-Tyr-Tyr-Pro-Trp-Pro-Val-Thr-Tyr-Lys-Gly-Gly-Gly-Lys(Biotin)-NH₂ |
| 3l | H-Leu-Phe-His-Pro-Trp-Pro-Ala-Phe-Pro-Lys-Gly-Gly-Gly-Lys(Biotin)-NH₂ |
| 3m | H-Leu-Trp-His-Pro-Trp-Ala-Met-Pro-Glu-Lys-Gly-Gly-Gly-Lys(Biotin)-NH₂ |
| 3n | H-Leu-Trp-Ser-Pro-Val-Phe-Phe-Phe-Pro-Lys-Gly-Gly-Gly-Lys(Biotin)-NH₂ |
| 3o | H-Met-Phe-His-Pro-Phe-Ala-Phe-Met-Tyr-Lys-Gly-Gly-Gly-Lys(Biotin)-NH₂ |
| 3p | H-Pro-Phe-Tyr-Thr-Tyr-His-Trp-Tyr-Ser-Lys-Gly-Gly-Gly-Lys(Biotin)-NH₂ |
| 3q | H-Pro-His-His-Pro-Phe-Ala-Leu-Phe-Leu-Lys-Gly-Gly-Gly-Lys(Biotin)-NH₂ |
| 3r | H-Ser-Gly-Val-Asp-Thr-Tyr-Leu-Thr-His-Lys-Gly-Gly-Gly-Lys(Biotin)-NH₂ |
| 3s | H-Val-Phe-His-Pro-Phe-Trp-Gly-Tyr-Phe-Lys-Gly-Gly-Gly-Lys(Biotin)-NH₂ |
| 3t | H-Trp-Val-Pro-Gly-Phe-Trp-Tyr-His-Tyr-Lys-Gly-Gly-Gly-Lys(Biotin)-NH₂ |
| 3u | H-Trp-Tyr-Leu-Pro-Pro-Leu-Trp-His-Ala-Lys-Gly-Gly-Gly-Lys(Biotin)-NH₂ |
| 3v | H-Tyr-Phe-Leu-Tyr-Leu-Pro-Tyr-His-Thr-Lys-Gly-Gly-Gly-Lys(Biotin)-NH₂ |
| 3w | H-Tyr-Trp-Val-Pro-Met-Trp-Val-His-Val-Lys-Gly-Gly-Gly-Lys(Biotin)-NH₂ |

In certain embodiments of any of the peptides disclosed in the above table, the biotin moiety is replaced with —Z, -L₂-Z, —S₁-L₂-Z, wherein Z, S₁, and L₂ are as defined herein.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., -CD₃, -OCD₃).

Any compound of the invention can also be radiolabed for the preparation of a radiopharmaceutical agent.

### Methods of Treatment

One aspect of the invention relates to a method of treating a disease, comprising administering to a subject in need thereof an effective amount of the conjugate of Formula I, wherein the conjugate comprises a drug moiety and the disease is the therapeutic target of the drug moiety.

In certain embodiments, the conjugate binds to erythrocytes in the subject.

In certain embodiments, the erythrocytes deliver the drug moiety to target cells in the subject.

In certain embodiments, administration of the conjugate extends the half-life of the linked drug as compared to administration of the free drug.

In certain embodiments, administration of the conjugate reduces the effectiveness of multidrug resistant (MDR) extrusion pumps in target cells in the subject as compared to administration of the free drug.

In certain embodiments, the conjugate bypasses multidrug resistant (MDR) extrusion pumps in the subject.

One aspect of the invention relates to a method of treating a disease, comprising administering to a subject in need thereof an effective amount of the conjugate of Formula I, wherein the conjugate comprises a GLP-1 agonist moiety and the disease is the therapeutic target of the GLP-1 agonist.

In certain embodiments, the conjugate binds to erythrocytes in the subject.

In certain embodiments, the erythrocytes deliver the GLP-1 agonist moiety to target cells in the subject.

In certain embodiments, the target cells are pancreatic islets.

In certain embodiments, the target cells are Beta cells (3-cells).

In certain embodiments, administration of the conjugate extends the half-life of the linked GLP-1 agonist as compared to administration of the free GLP-1 agonist.

In certain embodiments, the disease is a disease or disorder that is at least partially mediated by GLP-1 in a subject in need thereof comprising administering to the subject an effective amount of the conjugate of Formula I.

In certain embodiments, the disease or disorder that is at least partially mediated by GLP-1 is diabetes.

In certain embodiments, the diabetes is type-II diabetes.

In certain embodiments, a method of treating, preventing, or delaying the onset of complications related to diabetes, including macrovascular and microvascular complications such as retinopathy, neuropathy, nephropathy and delayed wound healing, and related diseases such as insulin resistance (impaired glucose homeostasis), hyperglycemia, hyperinsulinemia, elevated blood levels of fatty acids or glycerol, obesity, hyperlipidemia including hypertriglyceridemia, Syndrome X, atherosclerosis and hypertension, and for increasing high density lipoprotein levels.

In certain embodiments, the method further comprising administering an anti-diabetic agent.

In certain embodiments, the method further comprising administering a lipid lowering agent, which may be applied in the setting of human immunodeficiency virus (HIV) and its treatment.

In certain embodiments, a method of treating or preventing obesity or related metabolic disorders such as polycystic ovarian disease (PCOS) in a subject in need thereof comprising administering to the subject an effective amount of the conjugate of Formula I, wherein the conjugate comprises a GLP-1 agonist moiety.

In certain embodiments, the method further comprising administering an anti-obesity agent.

In certain embodiments, a method of treating or preventing cardiovascular disease in a subject in need thereof comprising administering to the subject an effective amount of the conjugate of Formula I, wherein the conjugate comprises a GLP-1 agonist moiety.

In certain embodiments, the method further comprising administering an anti-hypertensive agent.

In certain embodiments, a method of treating or preventing a neurodegenerative disease in a subject in need thereof comprising administering to the subject an effective amount of the conjugate of Formula I, wherein the conjugate comprises a GLP-1 agonist moiety.

In certain embodiments, the neurodegenerative disease is selected from Alzheimer's disease, Parkinson's disease, multiple sclerosis, amyotrophic lateral sclerosis, Huntington's disease, and prion diseases.

In certain embodiments, a method of treating or preventing a traumatic brain injury (TBI) in a subject in need thereof comprising administering to the subject an effective amount of the conjugate of Formula I, wherein the conjugate comprises a GLP-1 agonist moiety.

In certain embodiments, a method of treating or preventing non-alcoholic steatohepatitis (NASH) in a subject in need thereof comprising administering to the subject an effective amount of the conjugate of Formula I, wherein the conjugate comprises a GLP-1 agonist moiety.

Another aspect of the invention relates to a method of inducing antigen-specific immunotolerance, comprising administering to a subject in need thereof an effective amount of the conjugate of Formula I.

In certain embodiments, the conjugate binds to erythrocytes in the subject.

In certain embodiments, the conjugate decreases inflammatory antigen-specific T cell responses in the subject.

In certain embodiments, the conjugate decreased production of antigen-specific antibodies in the subject.

In certain embodiments, the conjugate decreases immunogenicity in the subject.

In certain embodiments, prior to producing immunotolerance, administration of the erythrocyte-targeted antigen to the subject results in an immune response in the subject.

In certain embodiments, the erythrocytes are mouse erythrocytes.

In certain embodiments, the erythrocytes are human erythrocytes.

In some embodiments of any one of the disclosed methods, the conjugate of Formula I is defined as:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)

wherein

R_{N} is the terminal H₂N— group of Xₐₐ¹;

Xₐₐ¹ is an optionally substituted D-Phe;

Xₐₐ² is an optionally substituted D-amino acid residue selected from D-Phe, D-Trp, D-Tyr, and D-Ala;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Phe, D-His, D-Lys, D-Met, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-amino acid residue selected from D-Lys and D-His;

R_{C} is the C-terminal —C(O)—NH— group of Xₐₐ¹⁰;

Y is the linker which links the peptide moiety and the active moiety; and

Z is the active moiety.

In some embodiments of any one of the disclosed methods, the conjugate of Formula I is defined as:

R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)

wherein

R_{N} is the terminal H₂N— group of Xₐₐ¹;

Xₐₐ¹ is an optionally substituted D-amino acid residue selected from D-Phe and D-Leu;

Xₐₐ² is an optionally substituted D-amino acid residue selected from D-Phe, D-Trp, D-Tyr, and D-Ala;

Xₐₐ³ is an optionally substituted D-amino acid residue selected from D-His, D-Leu, D-Nle, D-HNle, D-Abu, D-Cys, D-Ala, D-Met, D-Val, and D-Tyr;

Xₐₐ⁴ is an optionally substituted D-Pro, D-Ala, N-Me-D-Ala, and N-Me-Gly;

Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;

Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;

Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;

Xₐₐ⁸ is an optionally substituted D-amino acid residue selected from D-Phe, D-Ala, D-His, D-Lys, D-Met, D-Leu, D-Glu, D-Thr, D-Val, D-Trp, and D-Tyr;

Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;

Xₐₐ¹⁰ is an optionally substituted D-amino acid residue selected from D-Ala, D-Lys, and D-His;

R_{C} is the C-terminal —C(O)—NH— group of Xₐₐ¹⁰;

Y is the linker which links the peptide moiety and the active moiety; and

Z is the active moiety. In certain embodiments, Z is a drug moiety. In other embodiments, Z is a GLP-1 agonist moiety. In other embodiments, Z is an erythrocyte-targeted antigen moiety.

### Pharmaceutical Compositions, Routes of Administration, and Dosing

In certain embodiments, the invention is directed to a pharmaceutical composition, comprising a compound, i.e., conjugate, of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of, e.g., diabetes.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (a1-antitrypsin); Smith et al., 1989, J Clin Invest 84:1145-1146 (α-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (m), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Example 1. Synthesis of Peptide Dimer 1

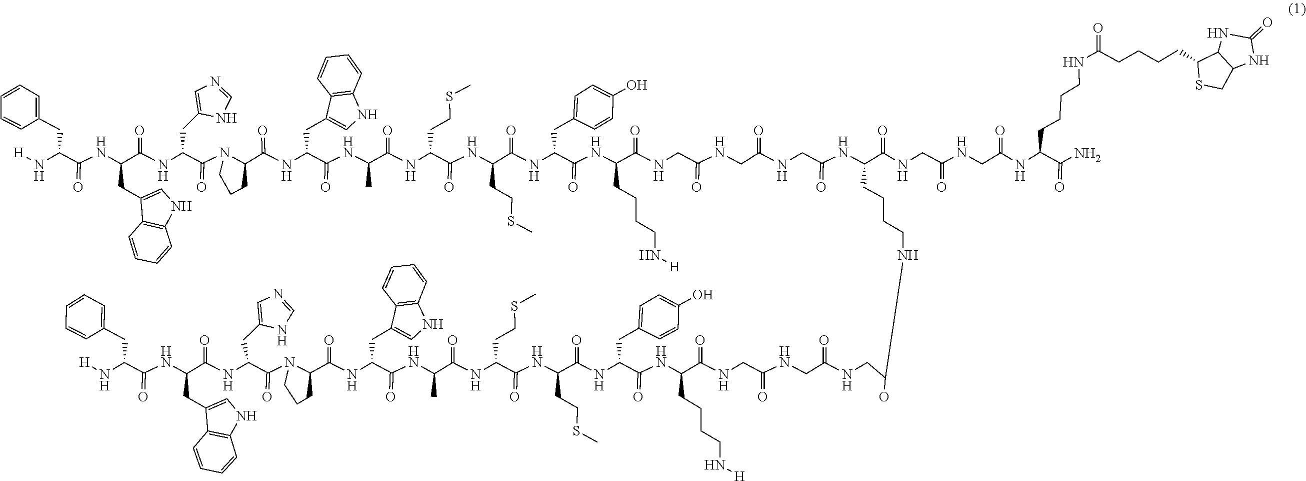

Synthesis—Dimer 1 was synthesized via Fmoc-based chemistry on a solid porous support on a Thuramed Tetras peptide synthesizer. The resin was Agilent Technologies AmphiSpheres 40 RAM resin, 75-150 μm, 0.37 mmol/g. Fmoc-D-Ala-OH, Fmoc-D-Phe-OH, Fmoc-Gly-OH, Fmoc-D-His(Trt)-OH, Fmoc-D-Lys(Boc)-OH, Fmoc-D-Met-OH, Fmoc-D-Pro-OH, Fmoc-D-Trp(Boc)-OH, and Fmoc-D-Tyr(tBu)—OH were double coupled (30 and 90 min, amino acids were 0.34 M, 5 eq.) with HCTU (0.5 M, 5 eq.), 6-Cl-HOBt (0.5 M, 5 eq.), and N,N-diisopropylethylamine (2 M, 10 eq.). This was followed by 9 washes of N,N-dimethylformamide. Amino acids, HCTU, 6-Cl-HOBt, and DIEA were dissolved in 1-methyl-2-pyrrolidinone. Fmoc-D-Lys(Fmoc)-OH (0.1M, 2 eq) and Fmoc-Lys(Biotin)-OH (0.1 M, 2 eq.) were coupled for 240 min with HATU (0.5 M, 2 eq.), HOAt (0.5 M, 2 eq.), and N,N-diisopropylethylamine (2 M, 4 eq.). This was followed by 9 washes of N,N-dimethylformamide. Amino acids, HATU, HOAt, and DIEA were dissolved in 1-methyl-2-pyrrolidinone. Fmoc was deprotected with 20% piperidine in DMF twice (10 and 15 min). This step was followed by 8 washes of N,N-dimethylformamide.Peptides were cleaved for two hours with the following cleavage cocktail: 5% water, 5% triisopropylsilane, 2.5% 1,2-ethanedithiol, 2.5% anisole, and 85% trifluoroacetic acid. Ten millimeters of cleavage cocktail was added per 1 gram of dry resin. Precipitated with cold diethyl ether, centrifuged at 3000 rpm, 5 minutes, and 0° C., and decanted the diethyl ether. Resuspended pellet with cold diethyl ether, centrifuged at 3000 rpm, 5 minutes, and 0° C., and decanted the diethyl ether. This was repeated one additional time. After the last decant step, the pellet was air dried.

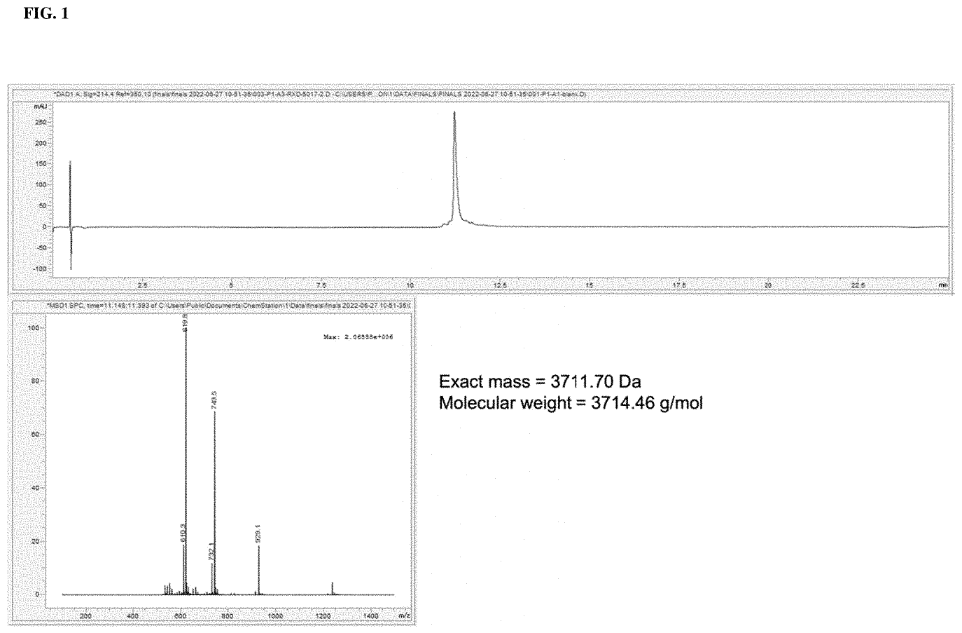

Purification—The crude material was purified on a Biotage Selekt. The column used for purification was a Biotage Sfär Bio C18 D-Duo 300 Å, 20 μm, 25 g. The flowrate was 40 mL/min. The A mobile phase was 0.1% trifluoroacetic acid in water. The B mobile phase was 0.1% trifluoroacetic acid in acetonitrile. Fractions were combined and lyophilized. Dry material was dissolved with 50% acetonitrile in water and lyophilized. This was repeated one additional time. FIG. 1 provides the LCMS characterization.

### Example 2. RBC Binding of Peptide Dimer 1

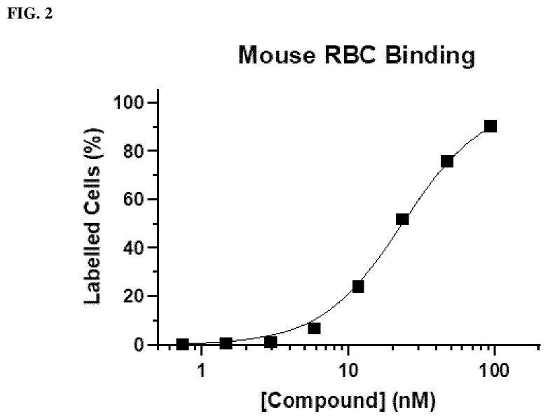

The peptide dimer 1 was resuspended in 100% DMSO, and then diluted to a desired starting concentration in 1×PBS. Peptide was then further diluted with 1×PBS to generate a concentration gradient. Washed mouse red cells (BioIVT, Westbury N.Y.) were prepared at a desired concentration of in 1×PBS. 5 volumes of red cell dilution were added to the peptide solution, and samples are incubated away from light at 4° C. with gentle shaking. The samples were centrifuged, and supernatants are removed by aspiration. Cell pellets were resuspended in 1×PBS containing streptavidin-APC (Abcam, Waltham, Mass.), and once again incubated away from light at 4° C. with gentle shaking. The samples were centrifuged once more, supernatants removed by aspiration, and cell pellets resupended in 1×PBS. Cells which have become fluorescently labelled by the streptavidin-APC were counted using flow cytometry (BD Accuri C6, BD Biosciences, Franklin Lake, N.J.) and a percent labelled cells value is determined. All samples were run in triplicate, error bars represent standard deviation. The peptide dimer 1 associated with the surface of RBCs in a concentration-dependent manner (FIG. 2).

### Example 3. Synthesis of GLP-1 Peptide with Albumin Binding Fatty Acid 2

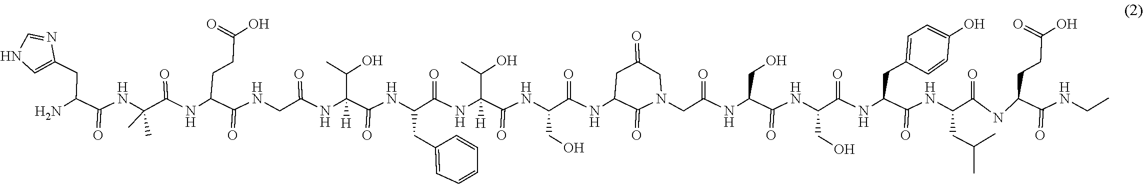

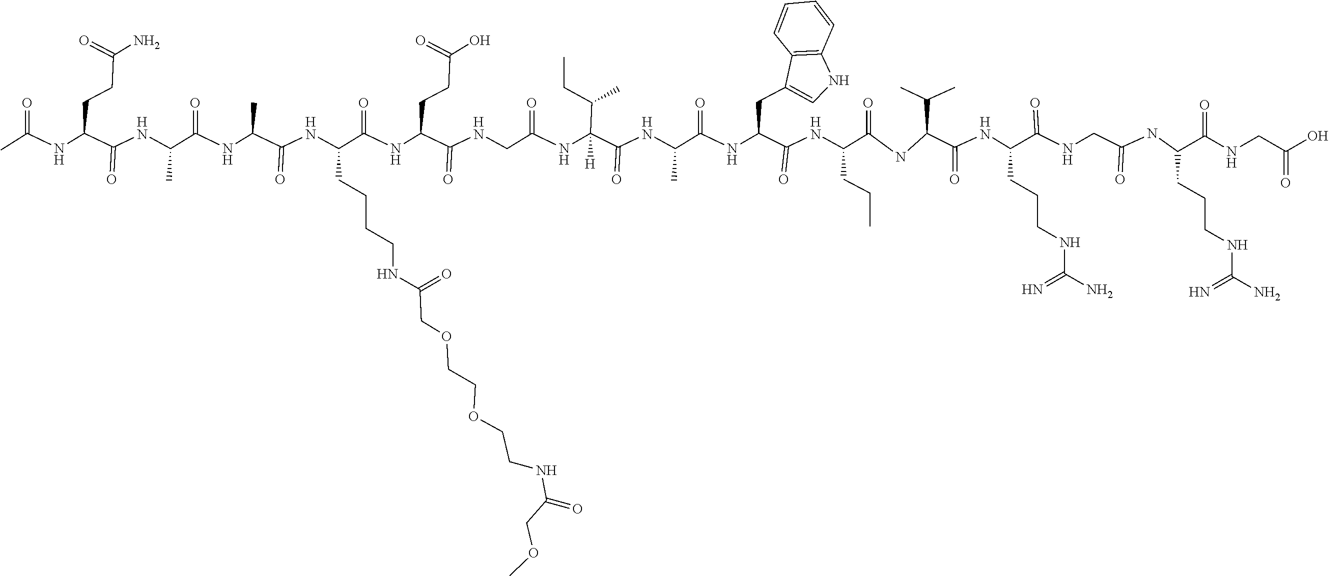

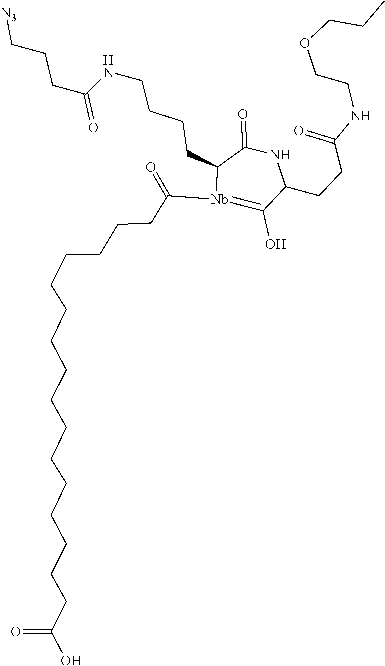

Synthesis—Peptide 2 was synthesized from a base peptide sequence obtained from a third party CRO. The peptide was received fully protected on-resin with an Fmoc-protected lysine side chain and a Boc-protected N-terminus. Synthesis was performed using Fmoc-based chemistry on-resin. Deprotection of the lysine side chain was performed by treating the resin with 20% piperidine in DMF for 10 mins at room temperature twice. After, the resin was washed 5× with DMF. Couplings were performed by preparing a solution of 0.55 M amino acid (5.5 eq), 0.5 M HATU (0.5 eq), HOAT (0.5 eq), and DIEA (1 M, 10 eq) in DMF. The solution was added to the resin and the reaction proceeded for 10 min. After, the resin was washed 5× with DMF. This process was used to install Fmoc-NH-PEG₂-CH₂COOH, Fmoc-NH-PEG₂-CH₂COOH, Fmoc-L-glutamic acid α-tert-butyl ester, and Fmoc-L-lysine(alloc). Fmoc deprotection was carried out once more to install octadecanoic acid mono-tert-butyl ester, which was prepared at a concentration of 0.28 M amino acid (5.5 eq), 0.25 M HATU (5 equiv), 0.25 M HOAt (5 eq), and 0.5 M DIEA (10 eq) in DMF. The reaction was allowed to proceed for 20 min. After the coupling, alloc deprotection was performed by transferring the resin to a vial under argon atmosphere. To this was added the deprotection solution: 0.3 equiv of tetrakis(triphenylphosphine)palladium(0) and 10 equivalents of morpholine (relative to peptide on resin) dissolved in DCM (10 mL/0.1 mmol peptide). The reaction was allowed to proceed for 30 mins and then was repeated once more. After, the resin was transferred to a peptide reaction vessel and washed 3× with DCM, 3×with 0.5% wt/wt DIEA in DMF, 3× with 0.5% wt/wt diethyldithiocarbamate, and 3×DMF. To this was coupled 4-azidobutyric acid. The peptide was cleaved for two hours with the following cleavage cocktail: 5% water, 5% triisopropylsilane, and 90% trifluoroacetic acid. Ten millimeters of cleavage cocktail was added per 1 gram of dry resin. Precipitated with cold diethyl ether, centrifuged at 3000 rpm, 5 minutes, and 0° C., and decanted the diethyl ether. Resuspended pellet with cold diethyl ether, centrifuged at 3000 rpm, 5 minutes, and 0° C., and decanted the diethyl ether. This was repeated one additional time. After the last decant step, the pellet was air dried.

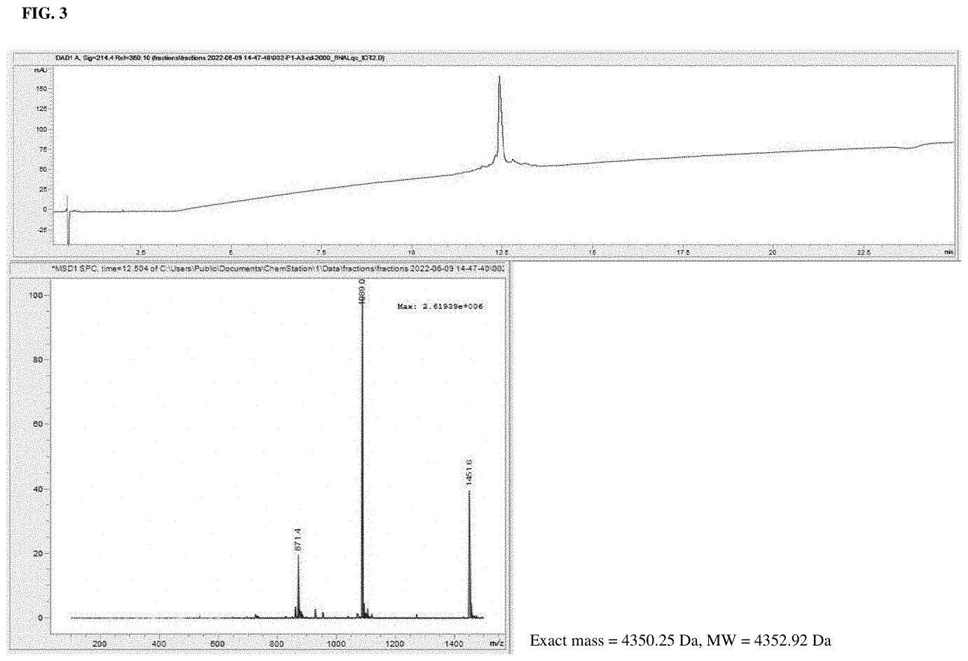

Purification—The crude material was purified on a Biotage Selekt. The column used for purification was a Biotage Sfär Bio C18 D-Duo 300 Å, 20 μm, 25 g. The flowrate was 40 mL/min. The A mobile phase was 0.1% trifluoroacetic acid in water. The B mobile phase was 0.1% trifluoroacetic acid in acetonitrile. Fractions were combined and lyophilized. Dry material was dissolved with 50% acetonitrile in water and lyophilized. This was repeated one additional time. FIG. 3 provides the LCMS characterization.

### Example 4. Synthesis of Intermediate Dimer 3

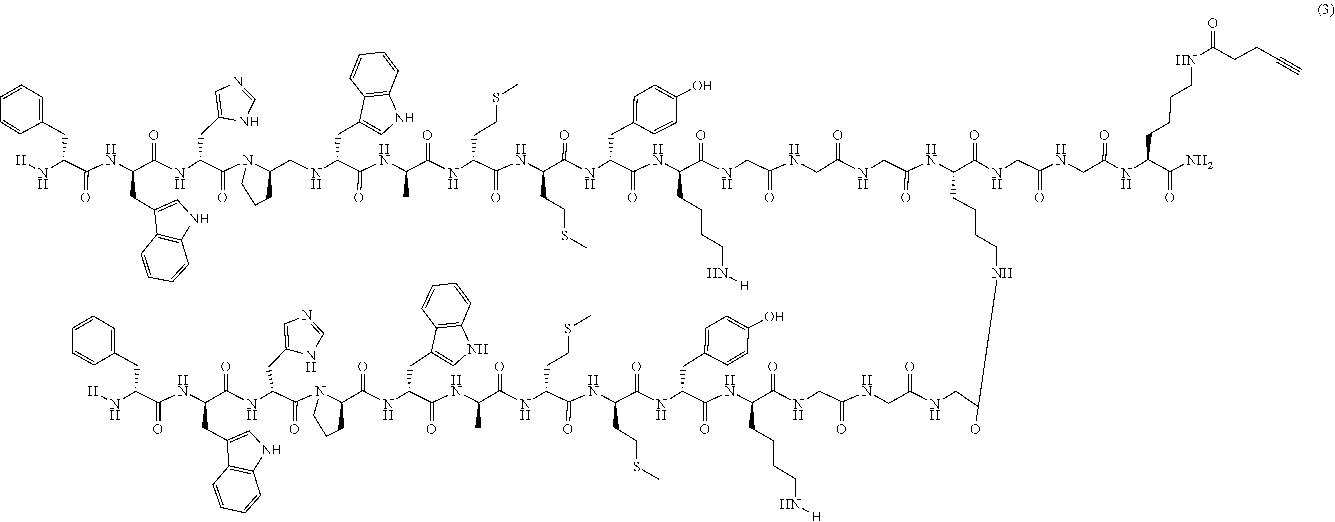

Synthesis—Dimer 3 was synthesized via Fmoc-based chemistry on a solid porous support. RXD-5043 was synthesized on a Thuramed Tetras peptide synthesizer. The resin was Agilent Technologies AmphiSpheres 40 RAM resin, 75-150 μm, 0.37 mmol/g. Fmoc-D-Ala-OH, Fmoc-D-Phe-OH, Fmoc-Gly-OH, Fmoc-D-His(Trt)-OH, Fmoc-D-Lys(Boc)-OH, Fmoc-D-Met-OH, Fmoc-D-Pro-OH, Fmoc-D-Trp(Boc)-OH, and Fmoc-D-Tyr(tBu)-OH were double coupled (30 and 90 min, amino acids were 0.34 M, 5 eq.) with HCTU (0.5 M, 5 eq.), 6-Cl-HOBt (0.5 M, 5 eq.), and N,N-diisopropylethylamine (2 M, 10 eq.). This was followed by 9 washes of N,N-dimethylformamide. Amino acids, HCTU, 6-Cl-HOBt, and DIEA were dissolved in 1-methyl pyrrolidinone. Fmoc-D-Lys(Fmoc)-OH (0.1M, 2 eq) and Fmoc-Lys(Mtt)-OH (0.1 M, 2 eq.) were coupled for 240 min with HATU (0.5 M, 2 eq.), HOAt (0.5 M, 2 eq.), and N,N-diisopropylethylamine (2 M, 4 eq.). This was followed by 9 washes of N,N-dimethylformamide. Amino acids, HATU, HOAt, and DIEA were dissolved in 1-methyl-2-pyrrolidinone. Fmoc was deprotected with 20% piperidine in DMF twice (10 and 15 min). This step was followed by 8 washes of N,N-dimethylformamide. The peptide was removed from the Tetras and the Mtt was deprotected by incubating the resin 6× for 2 min each time with 30% HFIP in DCM. After, 4-pentynoic acid was coupled onto the lysine side chain. The peptide was cleaved for two hours with the following cleavage cocktail: 5% water, 5% triisopropylsilane, 2.5% 1,2-ethanedithiol, 2.5% anisole, and 85% trifluoroacetic acid. Ten millimeters of cleavage cocktail was added per 1 gram of dry resin. Precipitated with cold diethyl ether, centrifuged at 3000 rpm, 5 minutes, and 0° C., and decanted the diethyl ether. Resuspended pellet with cold diethyl ether, centrifuged at 3000 rpm, 5 minutes, and 0° C., and decanted the diethyl ether. This was repeated one additional time. After the last decant step, the pellet was air dried.

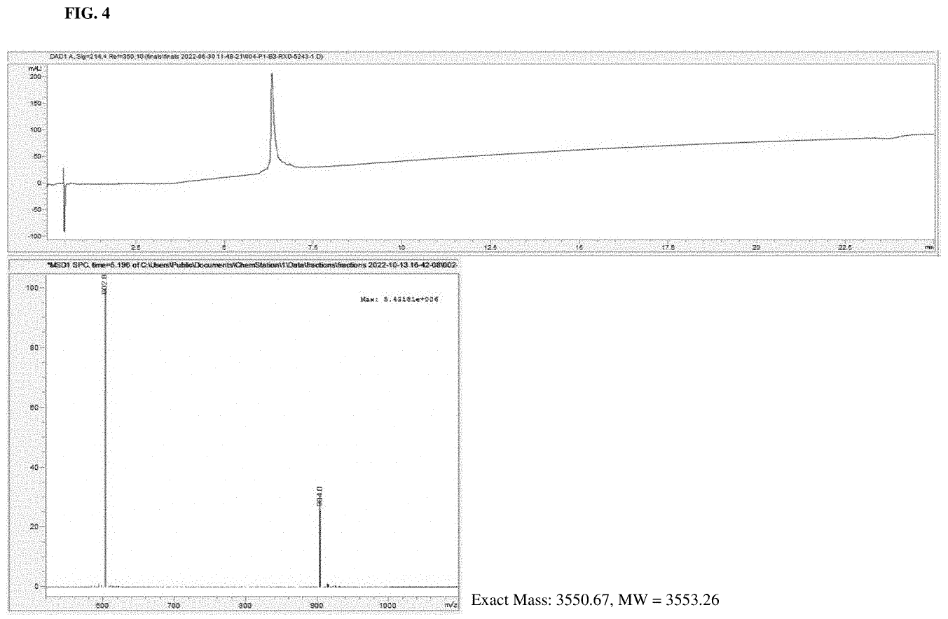

Purification—The crude material was purified on a Biotage Selekt. The column used for purification was a Biotage Sfär Bio C18 D-Duo 300 Å, 20 μm, 25 g. The flowrate was 40 mL/min. The A mobile phase was 0.1% trifluoroacetic acid in water. The B mobile phase was 0.1% trifluoroacetic acid in acetonitrile. Fractions were combined and lyophilized. Dry material was dissolved with 50% acetonitrile in water and lyophilized. This was repeated one additional time. FIG. 4 provides the LCMS characterization.

### Example 5. Synthesis of Peptide Dimer 4

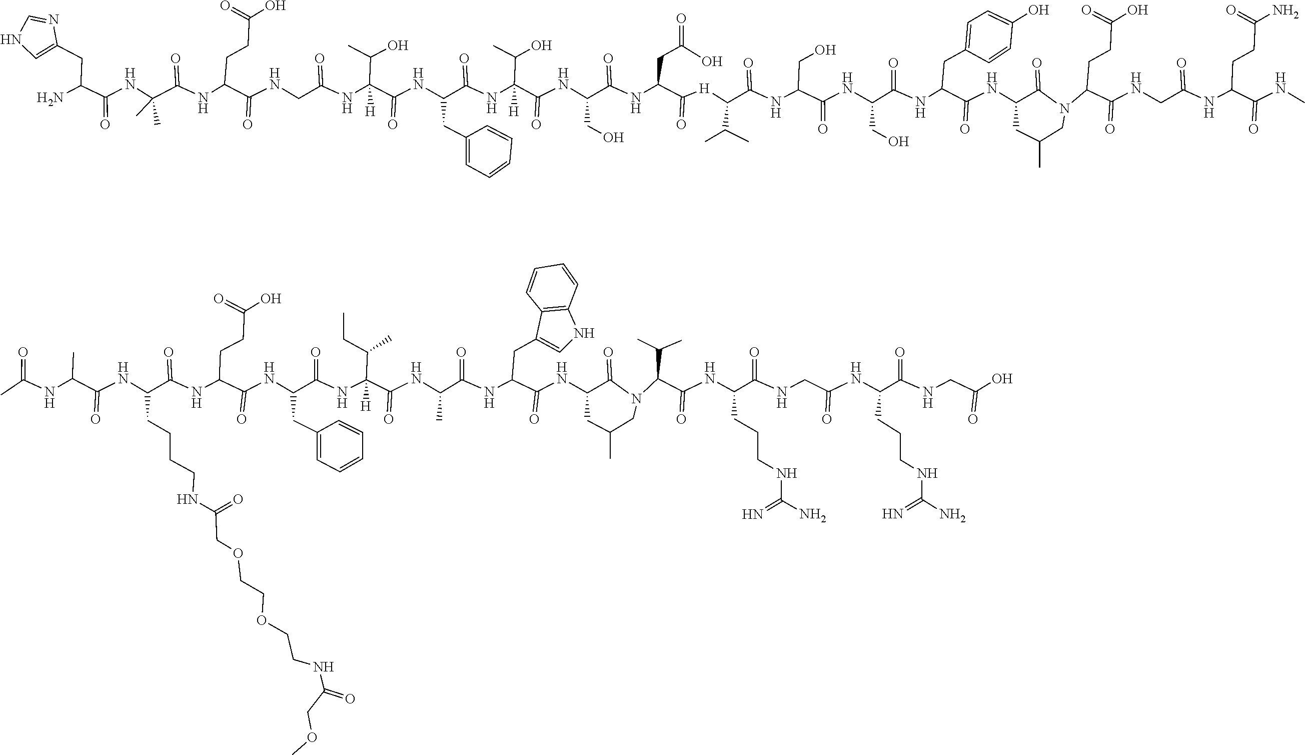

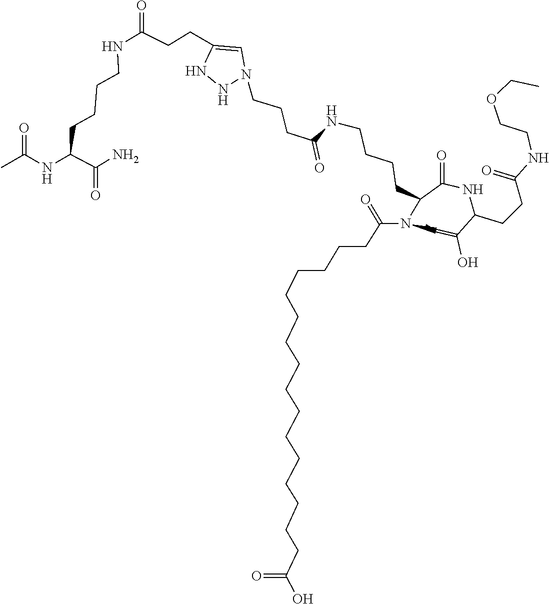

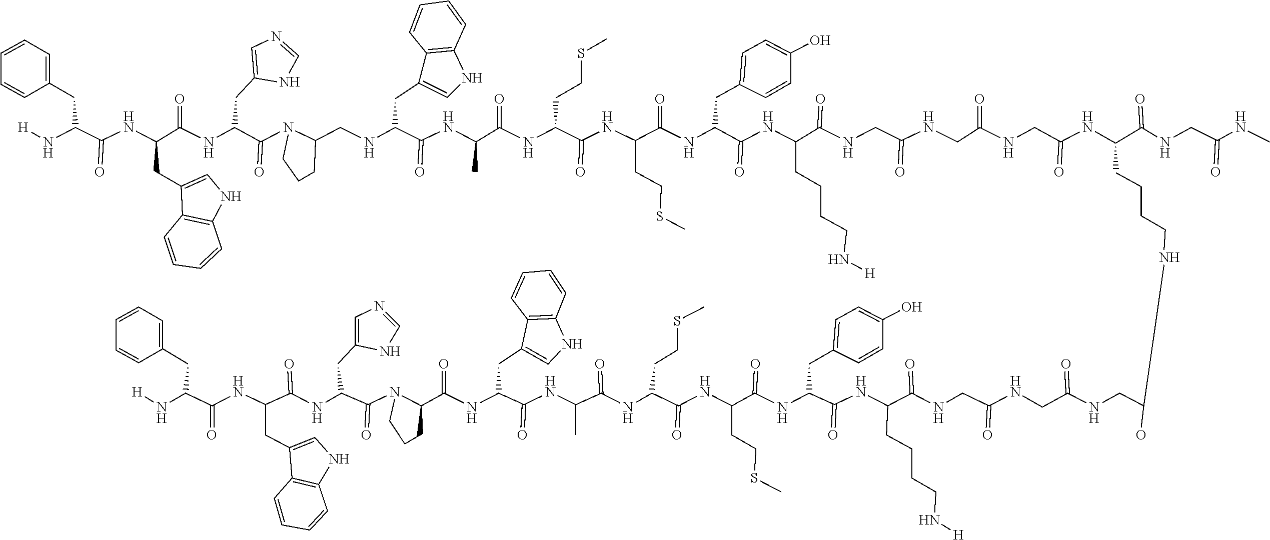

Synthesis—Dimer 4 was synthesized by combining 3 (1.2 eq, 1.2 mM), 2 (1 eq, 1 mM), CuI (5 eq, 5 mM), sodium ascorbate (5 eq, 5 mM), DIEA (25 eq, 25 mM) and 2,6-lutadine (0.2 eq, 0.2 mM) in DMSO. The reaction was allowed to proceed for 18 h at rt.

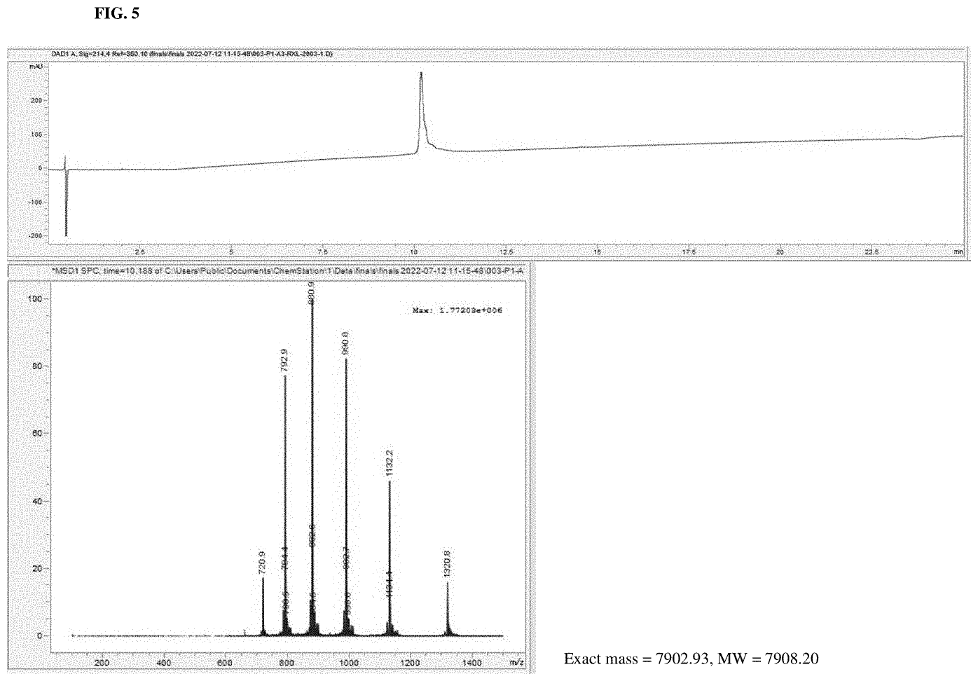

Purification—The crude material was purified on a Biotage Selekt. The column used for purification was a Biotage Sfär Bio C18 D-Duo 300 Å, 20 μm, 25 g. The flowrate was 40 mL/min. The A mobile phase was 0.1% trifluoroacetic acid in water. The B mobile phase was 0.1% trifluoroacetic acid in acetonitrile. Fractions were combined and lyophilized. Dry material was dissolved with 50% acetonitrile in water and lyophilized. This was repeated one additional time. FIG. 5 provides the LCMS characterization.

### Example 6. Synthesis of Antibody-Peptide Conjugate 6

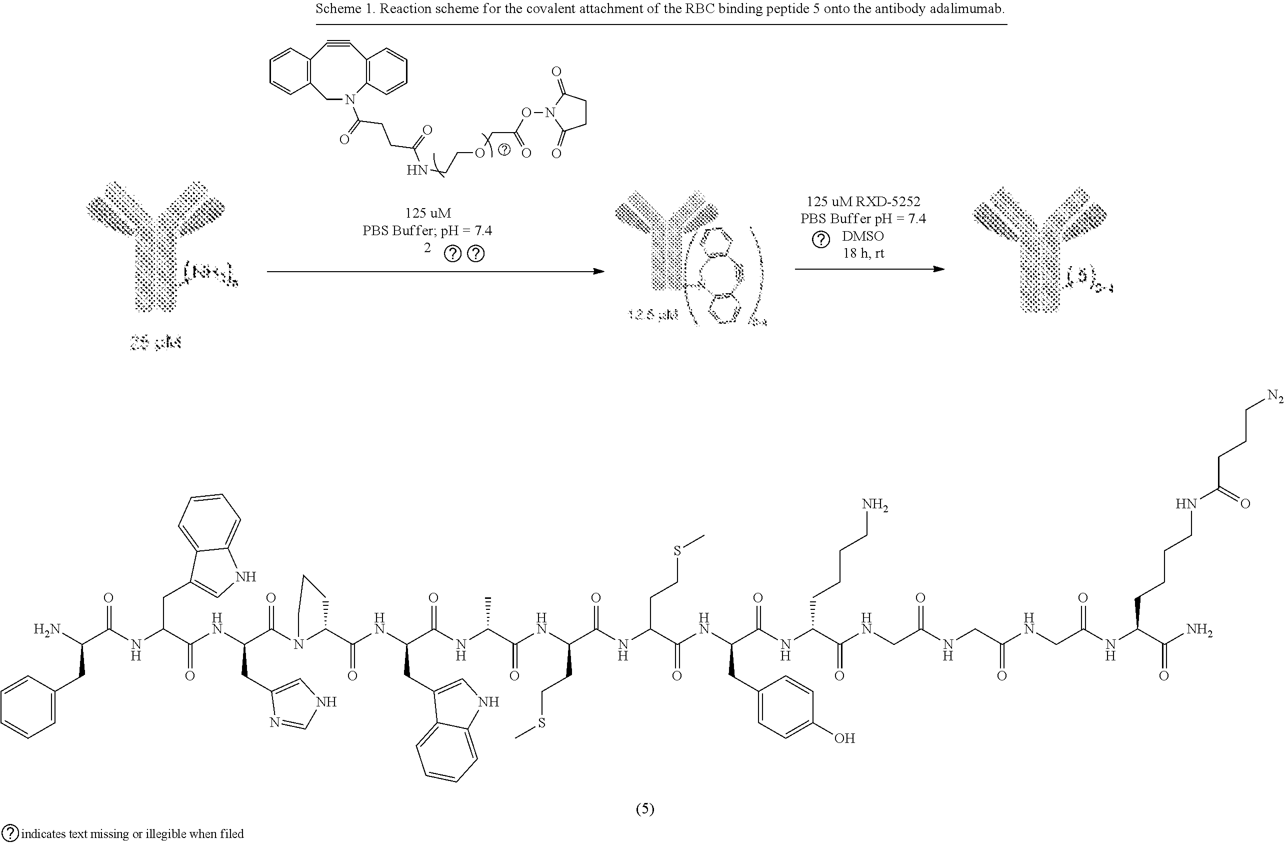

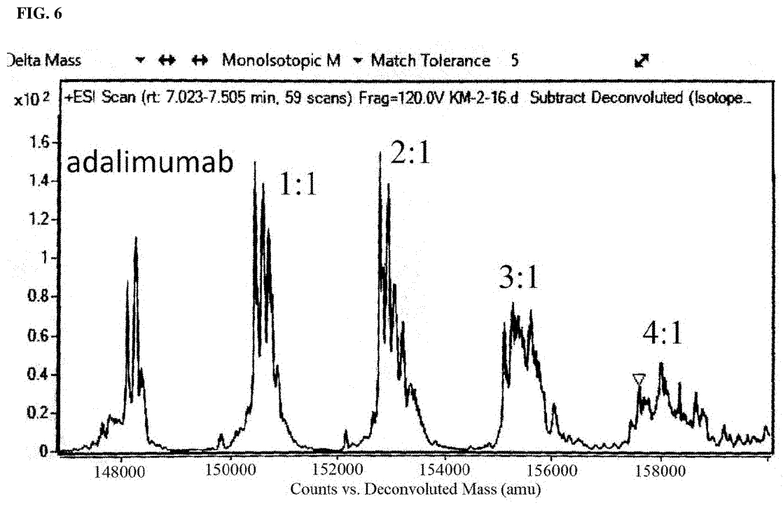

To a 1.5 mL Eppendorf tube was added phosphate buffered silane (PBS), adalimumab (ChemScene), and the desired amount of DBCO-PEG4-NHS ester (CAS: 1427004-19-0) as a stock in DMSO such that the final concentration was 125 uM. The solution was inverted twice to mix. This was allowed to react at room temperature for 2 h. After, PBS, DMSO, and the desired amount of Intermediate Peptide 5 as a DMSO stock was added and the reaction was allowed to proceed for 18 h at room temperature. The crude reaction mixture was analyzed by mass spectrometry and indicated that up to 4 peptides were covalently attached to the antibody adalimumab (FIG. 6).

### Example 7. RBC Binding of Antibody-Peptide Conjugate 6

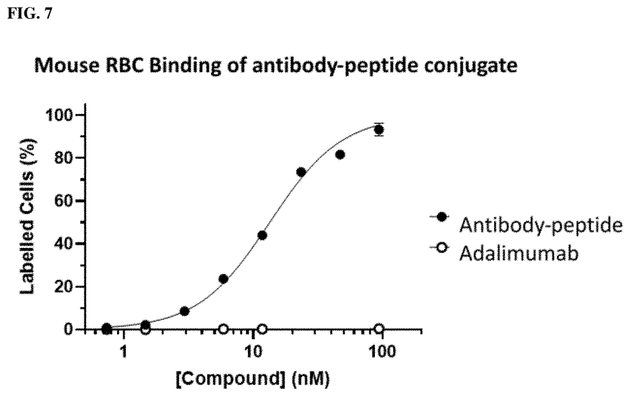

The antibody-peptide conjugate 6 was diluted to a desired starting concentration in 1×PBS. Conjugates were then further diluted with 1×PBS to generate a concentration gradient. Washed mouse red cells (BioIVT, Westbury N.Y.) were prepared at a desired concentration of in 1×PBS. 5 volumes of red cell dilution are added to the peptide solution, and samples were incubated away from light at 4 C with gentle shaking. The samples were centrifuged, and supernatants were removed by aspiration. Cell pellets were resuspended in 1×PBS containing PE-labelled Anti-Human IgG (Abcam, Waltham, Mass.), and once again were incubated away from light at 4° C. with gentle shaking. The samples were centrifuged once more, supernatants removed by aspiration, and cell pellets resupended in 1×PBS. Cells which have become fluorescently labelled by the PE-labelled secondary are counted using flow cytometry (BD Accuri C6, BD Biosciences, Franklin Lake, N.J.) and a percent labelled cells value was determined. All samples are run in triplicate, error bars represent standard deviation. Unconjugated adalimumab is shown as a control. The Antibody-Peptide Conjugate 6 associated with the surface of RBCs in a concentration-dependent manner (FIG. 7).

### Example 8. RBC Binding Properties of Exemplary D-Peptides

RBC binding ranked by MESF (molecules of equivalent soluble fluorescence), or intensity of fluorescent signal measured when RBCs were incubated with a given concentration of peptide.

High=MESF value>1000 fluorescent units

Medium=MESF Value from 300 to 1000 fluorescent units

Low=MESF Value from 15 to 300 fluorescent units

Does Not Bind=MESF Value below 15 fluorescent units”

TABLE 1
D-Ala scanning of 1a and classification of their RBC binding properties.

| No. | Peptide Sequence | Classification |
|---|---|---|
| 1a | H-phe-trp-his-pro-trp-ala-met-met-tyr-lys-Gly-Gly-Gly-lys(Biotin)-NH₂ | High |
| 1b | H-ala-trp-his-pro-trp-ala-met-met-tyr-lys-Gly-Gly-Gly-lys(Biotin)-NH₂ | Low |
| 1c | H-phe-ala-his-pro-trp-ala-met-met-tyr-lys-Gly-Gly-Gly-lys(Biotin)-NH₂ | Does Not Bind |
| 1d | H-phe-trp-ala-pro-trp-ala-met-met-tyr-lys-Gly-Gly-Gly-lys(Biotin)-NH₂ | Medium |
| 1e | H-phe-trp-his-ala-trp-ala-met-met-tyr-lys-Gly-Gly-Gly-lys(Biotin)-NH₂ | High |
| 1f | H-phe-trp-his-pro-ala-ala-met-met-tyr-lys-Gly-Gly-Gly-lys(Biotin)-NH₂ | Does Not Bind |
| ig | H-phe-trp-his-pro-trp-ala-ala-met-tyr-lys-Gly-Gly-Gly-lys(Biotin)-NH₂ | Does Not Bind |
| 1h | H-phe-trp-his-pro-trp-ala-met-ala-tyr-lys-Gly-Gly-Gly-lys(Biotin)-NH₂ | Low |
| 1i | H-phe-trp-his-pro-trp-ala-met-met-ala-lys-Gly-Gly-Gly-lys(Biotin)-NH₂ | Low |
| 1j | H-phe-trp-his-pro-trp-ala-met-met-tyr-ala-Gly-Gly-Gly-lys(Biotin)-NH₂ | High |

TABLE 2
1a analogs and classification of their RBC binding properties.

| RX ID | Peptide Sequence | Classification |
|---|---|---|
| 1a | H-phe-trp-his-pro-trp-ala-met-met-tyr-lys-Gly-Gly-Gly-lys(Biotin)-NH₂ | High |
| 2a | H-phe-trp-his-pro-trp-ala-nle-nle-tyr-lys-Gly-Gly-Gly-lys(Biotin)-NH₂ | Medium |
| 2b | H-phe-trp-his-pro-trp-ala-ala-ala-tyr-lys-Gly-Gly-Gly-lys(Biotin)-NH₂ | High |
| 2c | H-phe-trp-his-pro-trp-ala-leu-leu-tyr-lys-Gly-Gly-Gly-lys(Biotin)-NH₂ | Does Not Bind |
| 2d | H-phe-trp-his-pro-trp-ala-met-met-tyr-lys-Gly-Gly-Gly-(H-phe-trp-his- | High |
| | pro-trp-ala-met-met-tyr-lys-Gly-Gly-Gly)-lys-Gly-Gly-lys(Biotin)-NH₂ | |
| 2e | H-phe-trp-his-pro-trp-ala-met-met-tyr-lys-Gly-Gly-Gly-lys(C18diacid)- | Medium |
| | lys(Biotin)-NH₂ | |
| 2f | H-ala-pro-met-phe-tyr-trp-lys-his-met-trp-Gly-Gly-Gly-lys(Biotin)-NH₂ | Low |
| 2g | Ac-phe-trp-his-pro-trp-ala-met-met-tyr-lys-Gly-Gly-Gly-lys(Biotin)-NH₂ | Does Not Bind |
| 2h | H-phe-trp-his-pro-trp-ala-met-met-tyr-lys-Gly-Gly-Gly-cys(trt)-Gly- | Does Not Bind |
| | lys(Biotin)-NH₂ | |
| 2i | H-phe-trp-ser-pro-trp-ala-met-met-tyr-lys-Gly-Gly-Gly-lys(Biotin)-NH₂ | Low |
| 2j | H-phe-trp-tyr-pro-trp-ala-met-met-tyr-lys-Gly-Gly-Gly-lys(Biotin)-NH₂ | Low |
| 2k | H-phe-trp-his-NMeGly-trp-ala-met-met-tyr-lys-Gly-Gly-Gly-lys(Biotin)- | Low |
| | NH₂ | |
| 2l | H-phe-trp-his-L-Pro-trp-ala-met-met-tyr-lys-Gly-Gly-Gly-lys(Biotin)-NH₂ | Low |
| 2m | H-phe-trp-his-αMepro-trp-ala-met-met-tyr-lys-Gly-Gly-Gly-lys(Biotin)- | Does Not Bind |
| | NH₂ | |

TABLE 3
A series of structurally-diverse all-D-peptides of varying similarity to 1a and
classification of their RBC binding properties.

| RX ID | Peptide Sequence | Classification |
|---|---|---|
| 1a | H-phe-trp-his-pro-trp-ala-met-met-tyr-lys-Gly-Gly-Gly-lys(Biotin)-NH₂ | High |
| 3a | H-phe-phe-his-pro-trp-ser-trp-thr-ser-lys-Gly-Gly-Gly-lys(Biotin)-NH₂ | High |
| 3b | H-phe-phe-his-pro-tyr-Gly-leu-phe-val-lys-Gly-Gly-Gly-lys(Biotin)-NH₂ | Medium |
| 3c | H-phe-trp-his-pro-trp-phe-phe-lys-thr-lys-Gly-Gly-Gly-lys(Biotin)-NH₂ | Low |
| 3d | H-phe-trp-val-pro-phe-trp-trp-lys-thr-lys-Gly-Gly-Gly-lys(Biotin)-NH₂ | Low |
| 3e | H-leu-his-his-pro-trp-pro-tyr-phe-tyr-lys-Gly-Gly-Gly-lys(Biotin)-NH₂ | Low |
| 3f | H-leu-trp-his-pro-tyr-leu-phe-leu-trp-lys-Gly-Gly-Gly-lys(Biotin)-NH₂ | Does Not Bind |
| 3g | H-ser-trp-his-pro-val-trp-Gly-trp-leu-lys-Gly-Gly-Gly-lys(Biotin)-NH₂ | Low |
| 3h | H-phe-phe-his-pro-phe-ala-trp-phe-his-lys-Gly-Gly-Gly-lys(Biotin)-NH₂ | Low |
| 3i | H-phe-phe-his-val-pro-val-phe-ser-val-lys-Gly-Gly-Gly-lys(Biotin)-NH₂ | Medium |
| 3j | H-phe-trp-tyr-val-his-thr-thr-Gly-trp-lys-Gly-Gly-Gly-lys(Biotin)-NH₂ | Does Not Bind |
| 3k | H-phe-tyr-tyr-pro-trp-pro-val-thr-tyr-lys-Gly-Gly-Gly-lys(Biotin)-NH₂ | Does Not Bind |
| 3l | H-leu-phe-his-pro-trp-pro-ala-phe-pro-lys-Gly-Gly-Gly-lys(Biotin)-NH₂ | High |
| 3m | H-leu-trp-his-pro-trp-ala-met-pro-glu-lys-Gly-Gly-Gly-lys(Biotin)-NH₂ | Low |
| 3n | H-leu-trp-ser-pro-val-phe-phe-phe-pro-lys-Gly-Gly-Gly-lys(Biotin)-NH₂ | Does Not Bind |
| 3o | H-met-phe-his-pro-phe-ala-phe-met-tyr-lys-Gly-Gly-Gly-lys(Biotin)-NH₂ | Medium |
| 3p | H-pro-phe-tyr-thr-tyr-his-trp-tyr-ser-lys-Gly-Gly-Gly-lys(Biotin)-NH₂ | Does Not Bind |
| 3q | H-pro-his-his-pro-phe-ala-leu-phe-leu-lys-Gly-Gly-Gly-lys(Biotin)-NH₂ | Medium |
| 3r | H-ser-Gly-val-asp-thr-tyr-leu-thr-his-lys-Gly-Gly-Gly-lys(Biotin)-NH₂ | Medium |
| 3s | H-val-phe-his-pro-phe-trp-Gly-tyr-phe-lys-Gly-Gly-Gly-lys(Biotin)-NH₂ | Does Not Bind |
| 3t | H-trp-val-pro-Gly-phe-trp-tyr-his-tyr-lys-Gly-Gly-Gly-lys(Biotin)-NH₂ | Low |
| 3u | H-trp-tyr-leu-pro-pro-leu-trp-his-ala-lys-Gly-Gly-Gly-lys(Biotin)-NH₂ | Does Not Bind |
| 3v | H-tyr-phe-leu-tyr-leu-pro-tyr-his-thr-lys-Gly-Gly-Gly-lys(Biotin)-NH₂ | Low |
| 3w | H-tyr-trp-val-pro-met-trp-val-his-val-lys-Gly-Gly-Gly-lys(Biotin)-NH₂ | Low |

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A conjugate, comprising a peptide moiety, a linker, and an active moiety, having the following structure of formula (I):
R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-Xₐₐ¹⁰-R_{C}—Y—Z (I)
wherein
R_{N} is the terminal H₂N— group of Xₐₐ¹;
Xₐₐ¹ is an optionally substituted D-Phe;
Xₐₐ² is an optionally substituted D-amino acid residue selected from D-Phe, D-Trp, D-Tyr, and D-Ala;
Xₐₐ³ is an optionally substituted D-amino acid residue selected from D-His, D-Leu, D-Nle, D-HNle, D-Abu, D-Cys, D-Ala, D-Met, D-Val, and D-Tyr;
Xₐₐ⁴ is an optionally substituted D-Pro, D-Ala, N-Me-D-Ala, and N-Me-Gly;
Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;
Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;
Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;
Xₐₐ⁸ is an optionally substituted D-amino acid residue selected from D-Phe, D-Ala, D-His, D-Lys, D-Met, D-Leu, D-Glu, D-Thr, D-Val, D-Trp, and D-Tyr;
Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;
Xₐₐ¹⁰ is an optionally substituted D-amino acid residue selected from D-Lys and D-His;
R_{C} is the C-terminal —C(O)—NH— group of Xₐₐ¹⁰;
Y is the linker which links the peptide moiety and the active moiety; and
Z is the active moiety.

2. The conjugate of claim 1, wherein
R_{N} is the terminal H₂N— group of Xₐₐ¹;
Xₐₐ¹ is an optionally substituted D-Phe;
Xₐₐ² is an optionally substituted D-amino acid residue selected from D-Phe, D-Trp and D-Tyr;
Xₐₐ³ is an optionally substituted D-amino acid residue selected from D-His, D-Leu, D-Nle, D-HNle, D-Abu, D-Cys, D-Ala, D-Met, D-Val, and D-Tyr;
Xₐₐ⁴ is an optionally substituted D-Pro, D-Ala, N-Me-D-Ala, and N-Me-Gly;
Xₐₐ⁵ is an optionally substituted D-amino acid residue selected from D-Phe, D-His, D-Leu, D-Met, D-Pro, D-Ala, N-Me-D-Ala, N-Me-Gly, D-Val, D-Trp, D-Tyr, and N-Me-Gly;
Xₐₐ⁶ is an optionally substituted D-amino acid residue selected from D-Ala, D-Phe, D-Leu, D-Met, D-Nle, D-HNle, D-Abu, D-Cys, D-Pro, N-Me-D-Ala, N-Me-Gly, D-Ser, D-Thr, D-Val, D-Trp, and D-Tyr;
Xₐₐ⁷ is an optionally substituted D-amino acid residue selected from D-Ala, D-Phe, D-Leu, D-Met, D-Nle, D-HNle, D-Abu, D-Cys, D-Trp, and D-Tyr;
Xₐₐ⁸ is an optionally substituted D-amino acid residue selected from D-Phe, D-His, D-Lys, D-Leu, D-Glu, D-Thr, D-Val, D-Trp, and D-Tyr;
Xₐₐ⁹ is an optionally substituted D-amino acid residue selected from D-Phe, D-His, D-Leu, D-Pro, D-Ala, N-Me-D-Ala, N-Me-Gly, D-Ser, D-Glu, D-Thr, D-Val, D-Trp, and D-Tyr;
Xₐₐ¹⁰ is an optionally substituted D-Lys;
R_{C} is the C-terminal —C(O)—NH— group of Xₐₐ¹⁰;
Y is the linker which links the peptide moiety and the active moiety; and
Z is the active moiety.

3. The conjugate of claim 1, wherein
Xₐₐ³ is an optionally substituted D-amino acid residue selected from D-His, D-Leu, D-Met, D-Val, and D-Tyr;
Xₐₐ⁵ is an optionally substituted D-amino acid residue selected from D-Phe, D-His, D-Leu, D-Met, D-Pro, D-Val, D-Trp, and D-Tyr;
Xₐₐ⁶ is an optionally substituted D-amino acid residue selected from D-Ala, D-Phe, D-Leu, D-Met, D-Pro, D-Ser, D-Thr, D-Val, D-Trp, and D-Tyr;
Xₐₐ⁷ is an optionally substituted D-amino acid residue selected from D-Ala, D-Phe, D-Leu, D-Met, D-Trp, and D-Tyr; and
Xₐₐ⁹ is an optionally substituted D-amino acid residue selected from D-Phe, D-His, D-Leu, D-Pro, D-Ser, D-Glu, D-Thr, D-Val, D-Trp, and D-Tyr; or
Xₐₐ⁵ is an optionally substituted D-amino acid residue selected from D-Phe, D-His, D-Leu, D-Met, D-Pro, D-Ala, D-Val, D-Trp, and D-Tyr;
Xₐₐ⁶ is an optionally substituted D-amino acid residue selected from D-Ala, D-Phe, D-Leu, D-Met, D-Nle, D-HNle, D-Abu, D-Cys, D-Pro, D-Ser, D-Thr, D-Val, D-Trp, and D-Tyr; and
Xₐₐ⁹ is an optionally substituted D-amino acid residue selected from D-Phe, D-His, D-Leu, D-Pro, D-Ala, D-Ser, D-Glu, D-Thr, D-Val, D-Trp, and D-Tyr.

4. (canceled)

5. The conjugate of claim 1, wherein Xₐₐ¹ is unsubstituted D-Phe; and/or
Xₐₐ² is an unsubstituted D-amino acid residue selected from D-Phe, D-Trp and D-Tyr; and/or
Xₐₐ³ is an unsubstituted D-amino acid residue selected from D-His, D-Leu, D-Met, D-Val, and D-Tyr; and/or
Xₐₐ⁴ is D-Pro; and/or
Xₐₐ⁵ is an unsubstituted D-amino acid residue selected from D-Phe, D-His, D-Leu, D-Met, D-Pro, D-Val, D-Trp, and D-Tyr; and/or
Xₐₐ⁶ is an unsubstituted D-amino acid residue selected from D-Ala, D-Phe, D-Leu, D-Met, D-Pro, D-Ser, D-Thr, D-Val, D-Trp, and D-Tyr; and/or
Xₐₐ⁷ is an unsubstituted D-amino acid residue selected from D-Ala, D-Phe, D-Leu, D-Met, D-Trp, and D-Tyr; and/or
Xₐₐ⁸ is an unsubstituted D-amino acid residue selected from D-Phe, D-His, D-Lys, D-Met, D-Leu, D-Glu, D-Thr, D-Val, D-Trp, and D-Tyr; and/or
Xₐₐ⁹ is an unsubstituted D-amino acid residue selected from D-Phe, D-His, D-Leu, D-Pro, D-Ser, D-Glu, D-Thr, D-Val, D-Trp, and D-Tyr; and/or
Xₐₐ¹⁰ is an unsubstituted or substituted D-Lys or an unsubstituted or substituted D-His.

6.-26. (canceled)

27. The conjugate of claim 1, having the structure:

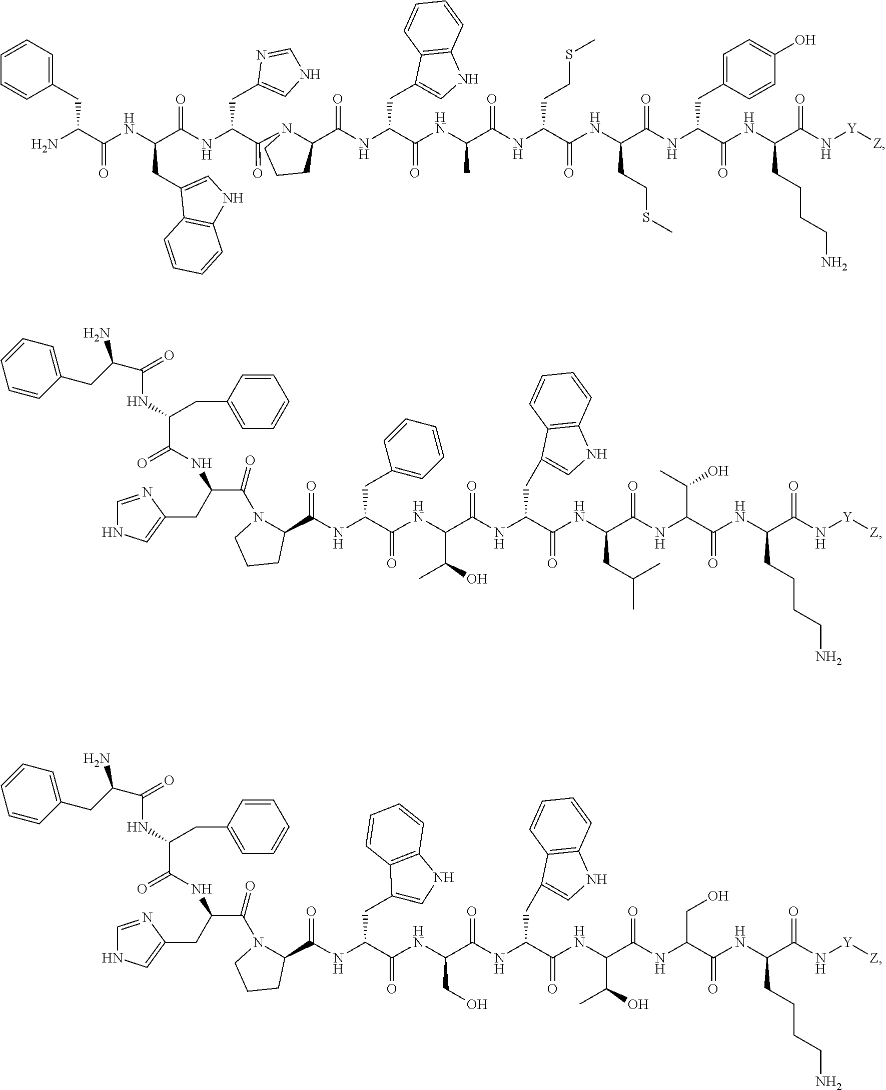

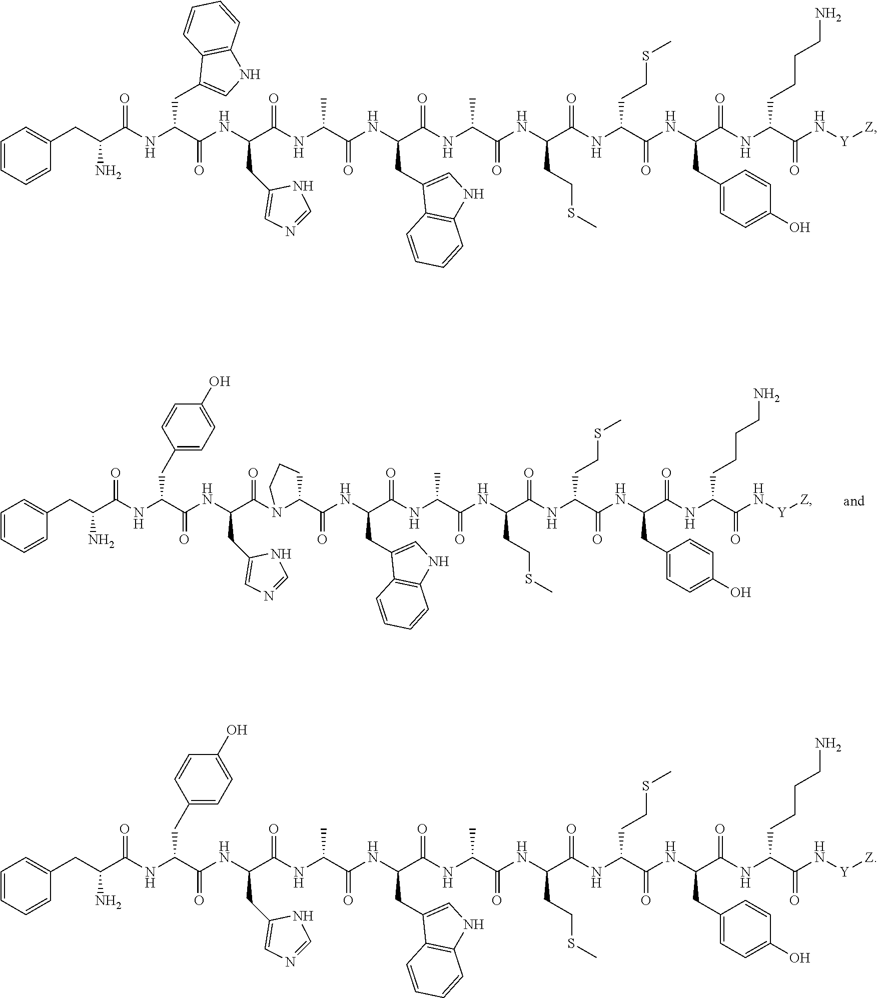

28.-29. (canceled)

30. The conjugate of claim 1, wherein Y has the structure -L₁-, -L₁-L₂-, or -L₁-S₁-L₂-
wherein
L₁ is a first linker unit;
S₁ is a spacer; and
L₂ is a second linker unit.

31.-35. (canceled)

36. The conjugate of claim 30, wherein L₁ is a peptide which comprises at least one internal substituted D-Lys or D-Cys.

37. (canceled)

38. The conjugate of claim 36, wherein the sidechain amino group of the internal D-Lys or the sidechain thio group of the internal D-Cys is substituted with a second peptide of claim 1.

39. The conjugate of claim 38, wherein the second peptide has the structure:
R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-
Xₐₐ¹⁰-R_{C}-L₁ [symbol]
wherein L₁′ is a third linker unit.

40.-42. (canceled)

43. The conjugate of claim 39, wherein L₁′ is a peptide which further comprises at least one optionally substituted D-Lys or D-Cys.

44. The conjugate of claim 43, wherein L₁′ terminates at the C-terminus with a Gly, D-Lys, or D-Cys which is bonded to the internal D-Lys or internal D-Cys of L₁.

45. The conjugate of claim 44, wherein the sidechain amino group of the C-terminal D-Lys of L₁′ or the sidechain thio group of the C-terminal D-Cys of L₁′ is substituted with a third peptide of claim 1.

46. The conjugate of claim 45, wherein the third peptide has the structure:
R_{N}-Xₐₐ¹-Xₐₐ²-Xₐₐ³-Xₐₐ⁴-Xₐₐ⁵-Xₐₐ⁶-Xₐₐ⁷-Xₐₐ⁸-Xₐₐ⁹-
Xₐₐ¹⁰-R_{C}-L₁″- [symbol]
wherein L₁″ is a fourth linker unit, optionally wherein L₁″ is a peptide.

47.-52. (canceled)

53. The conjugate of claim 30, wherein S₁ is present; and the sidechain amino group of the terminal D-Lys of L₁ is bonded to S₁.

54.-58. (canceled)

59. The conjugate of claim 53, wherein L₂ comprises a succinimide moiety, a 1,2,3-triazole moiety, or a PEG moiety.

60.-90. (canceled)

91. The conjugate of claim 1, wherein Z is a drug moiety, an erythrocyte-targeted antigen moiety, from an antibody, virus, hormone, enzyme, peptide, protein, or single-chain variable fragment (scFv), immunoglobulin G (IgG), adeno-associated virus (AAV), or human growth hormone (HGH, insulin, or a biotin moiety.

92.-108. (canceled)

109. The conjugate of claim 30, wherein the conjugate has the structure:

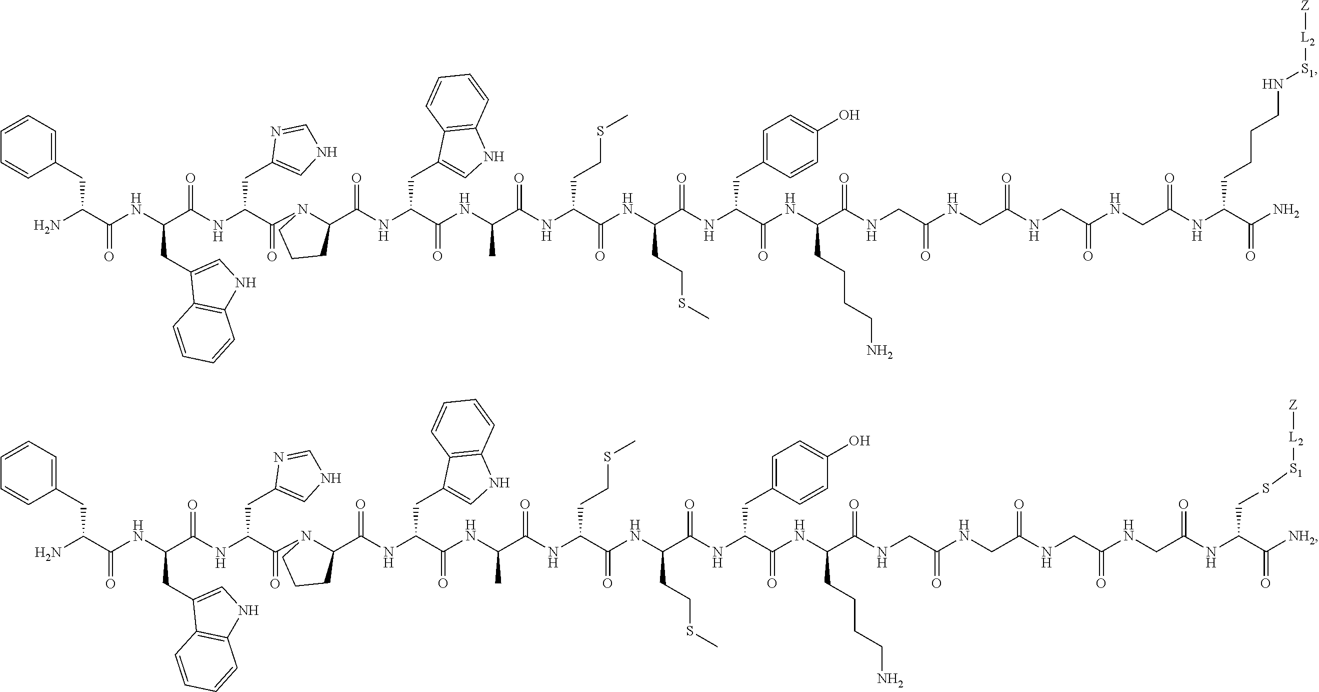

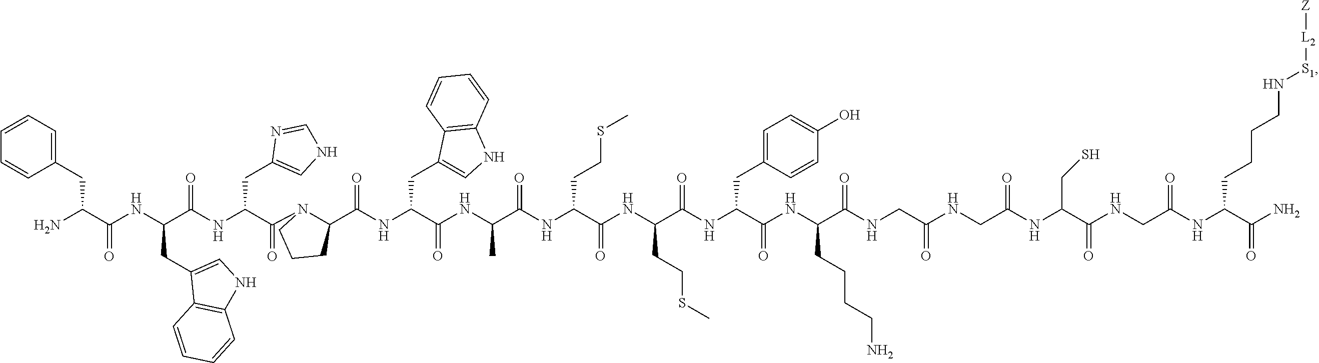

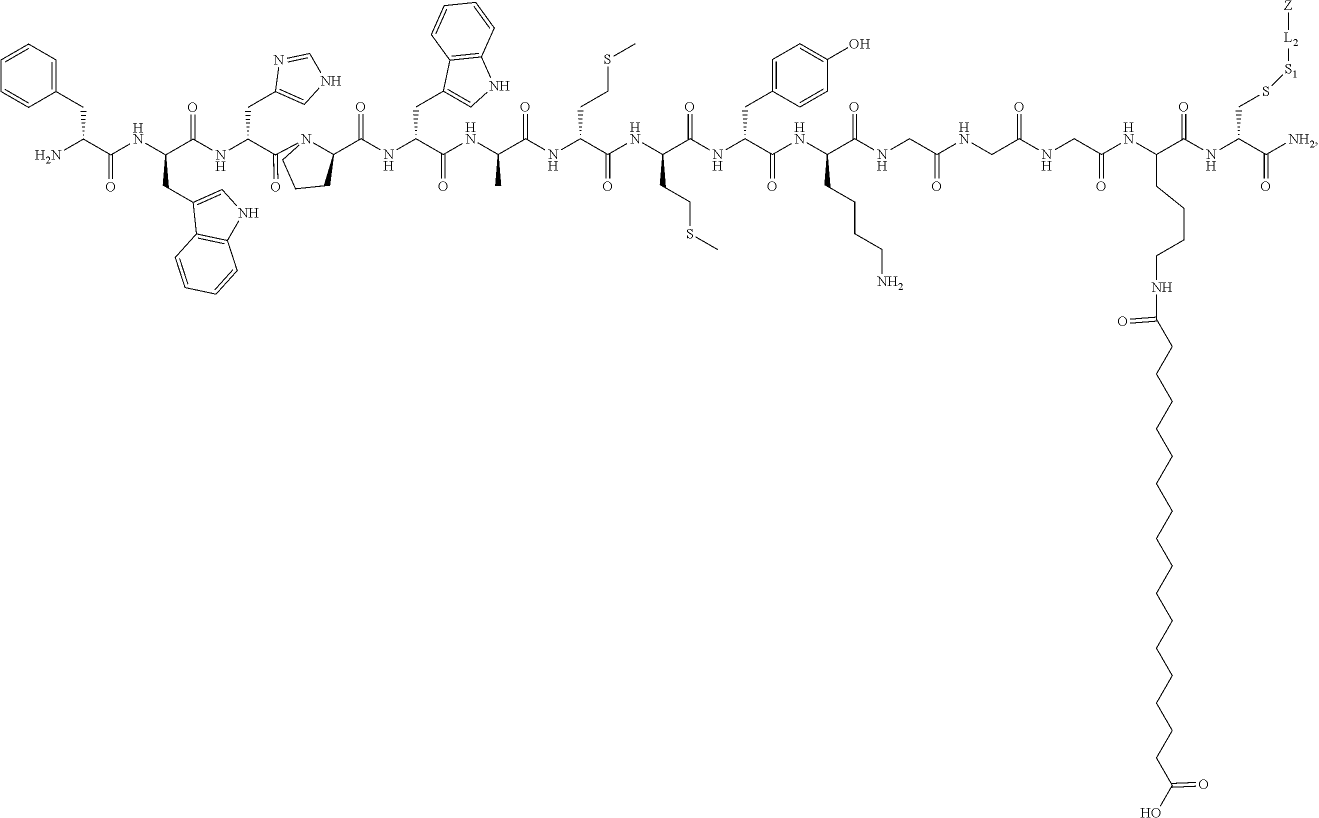

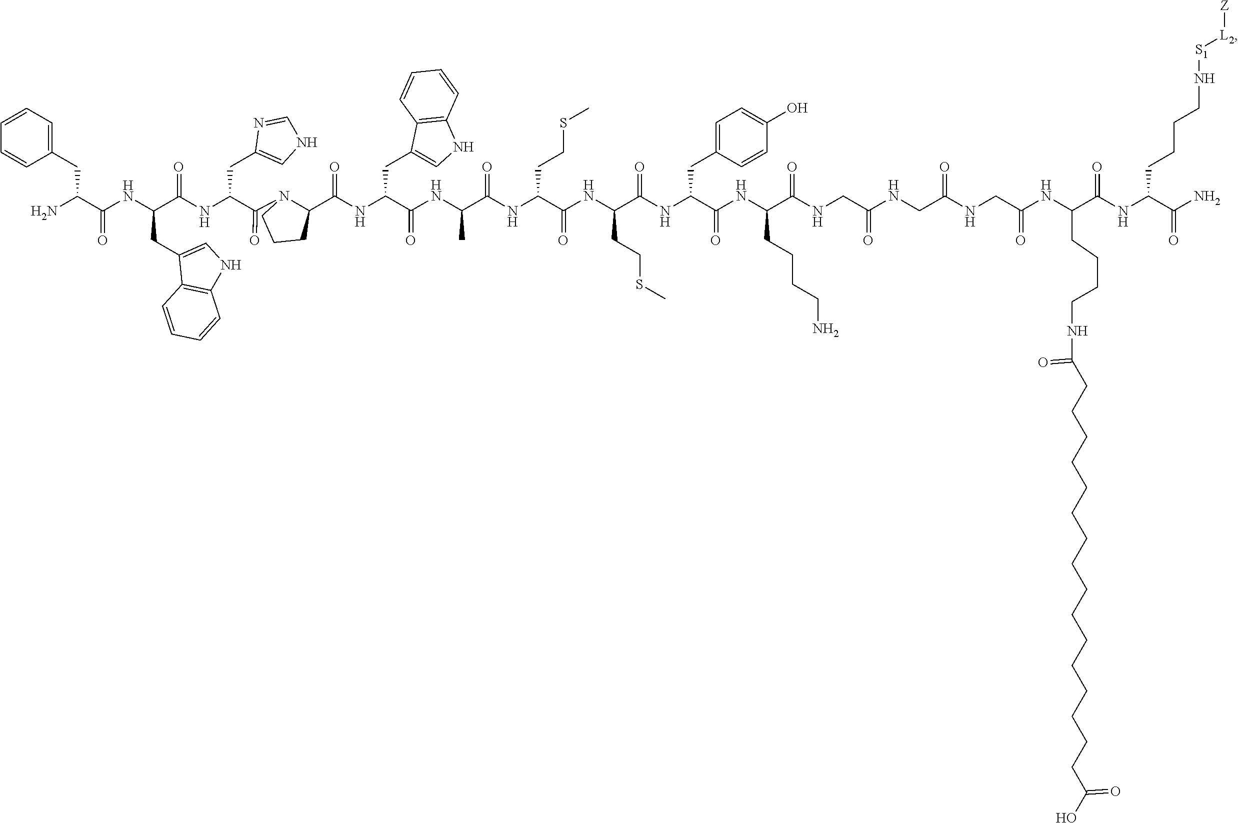

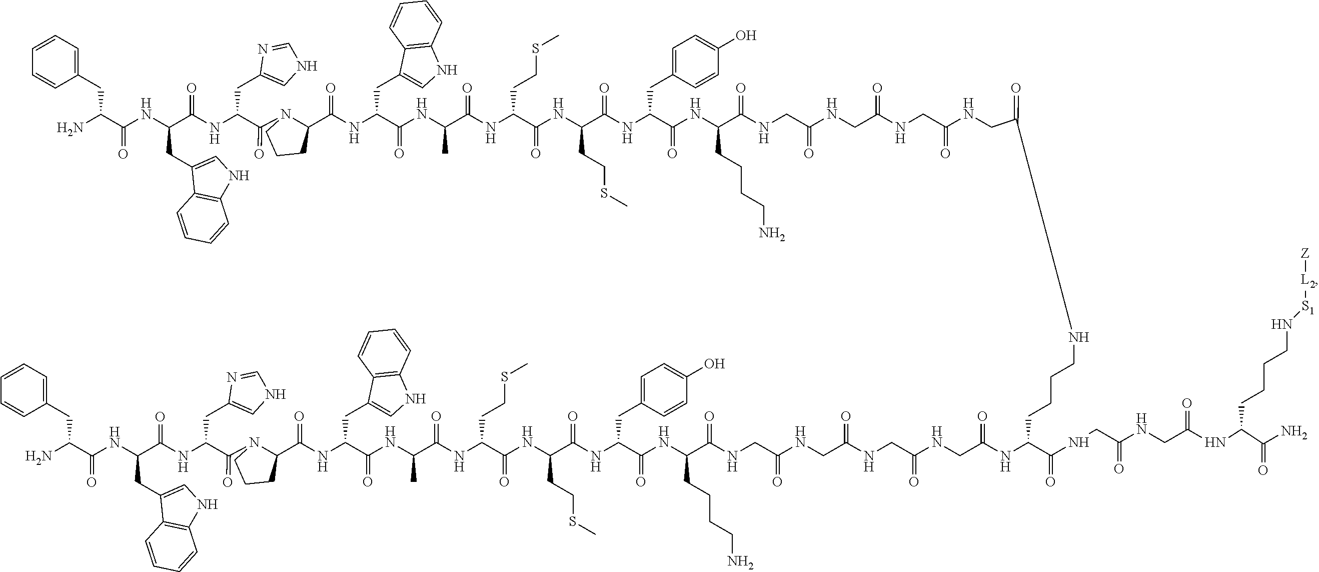

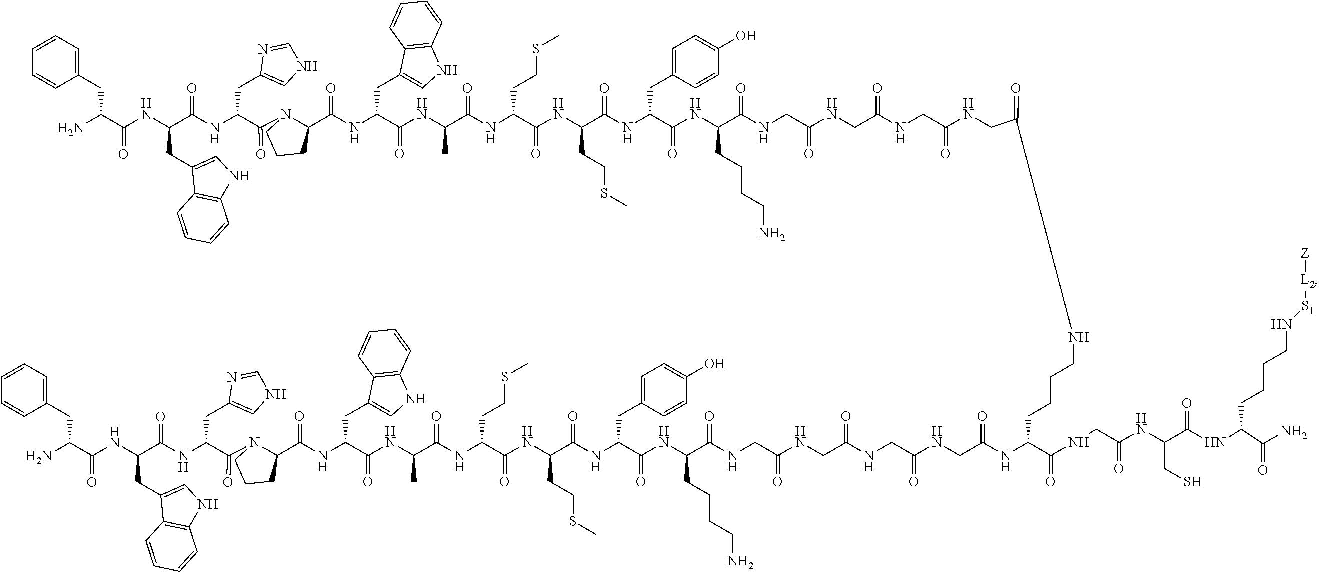

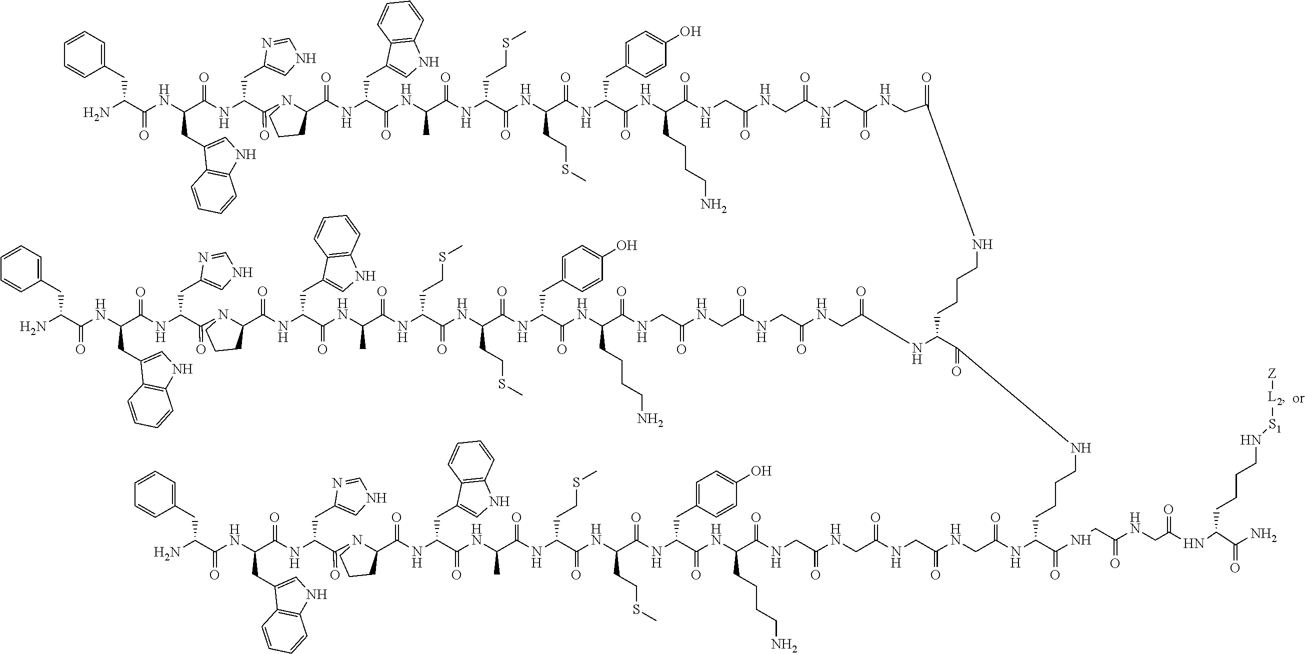

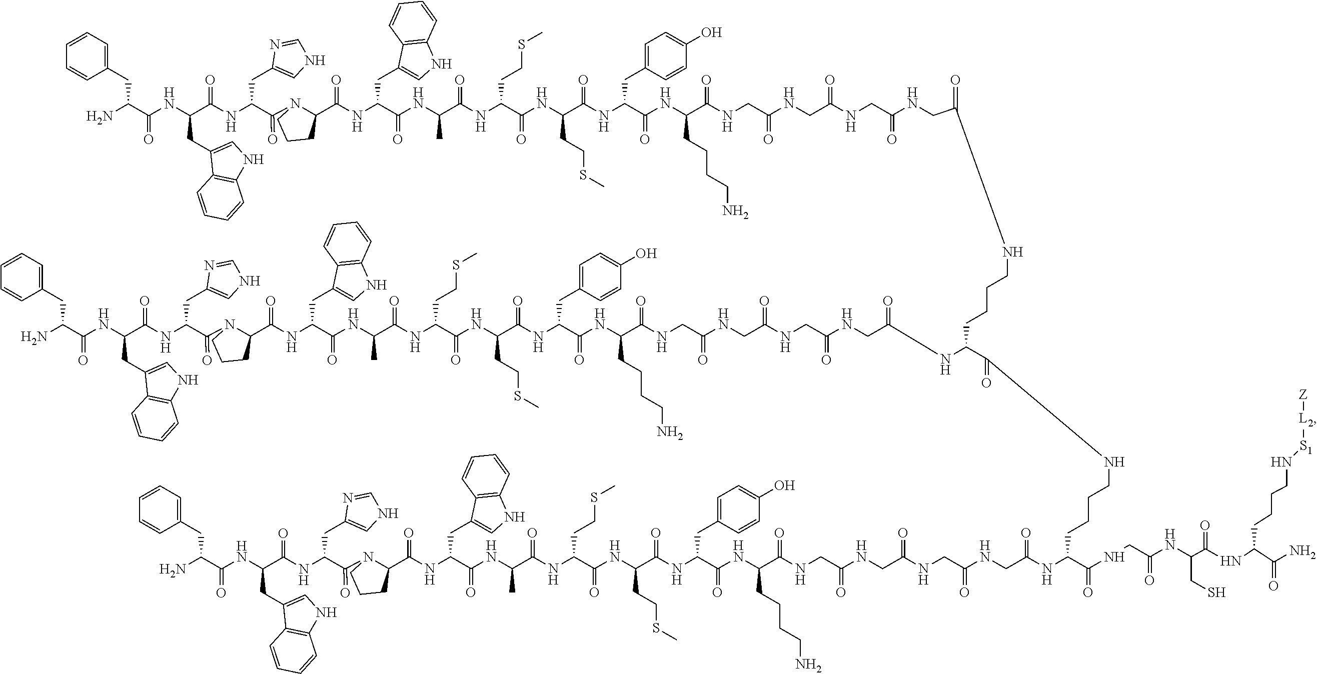

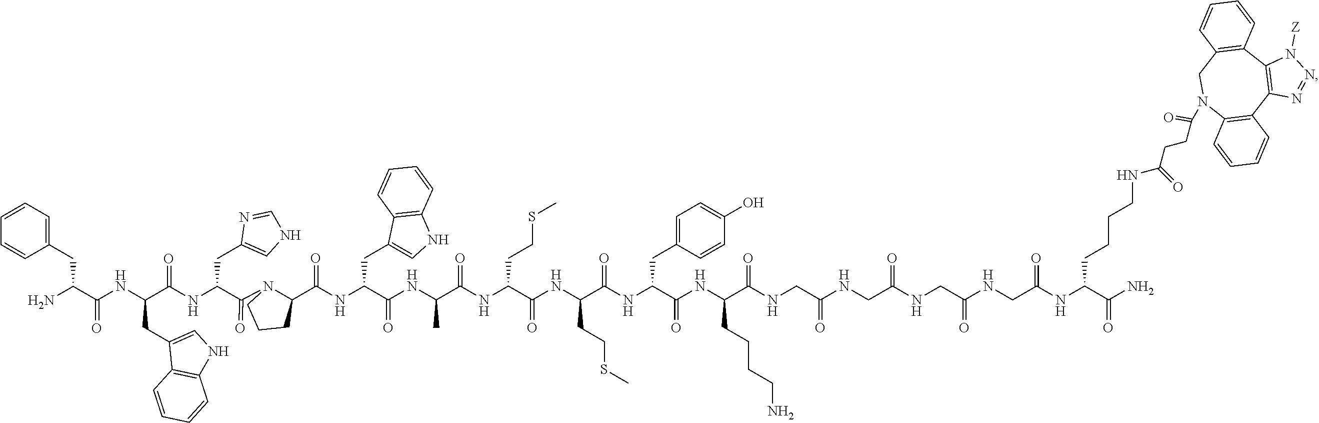

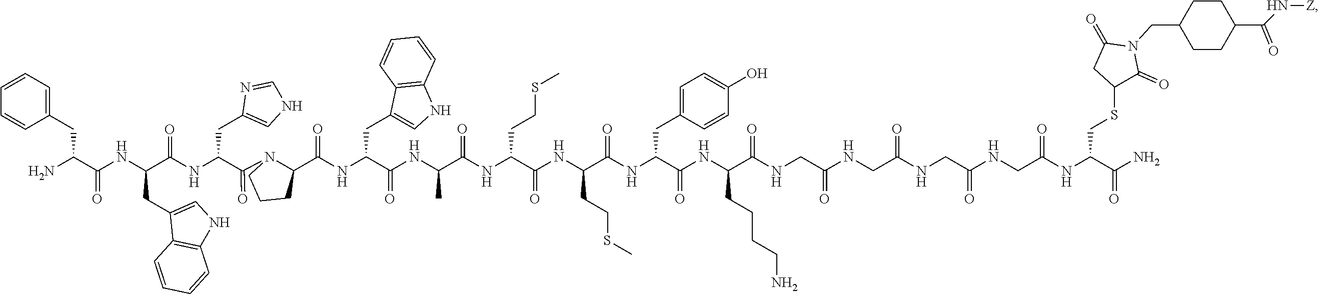

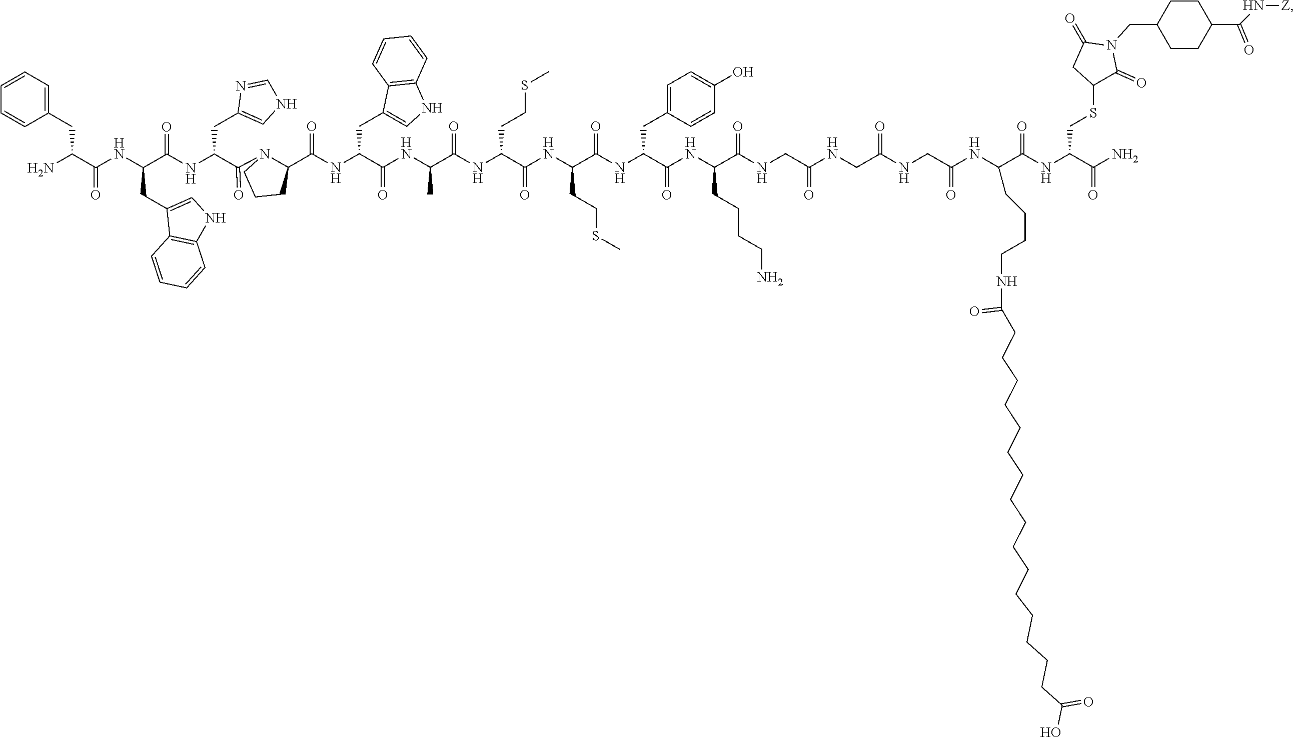

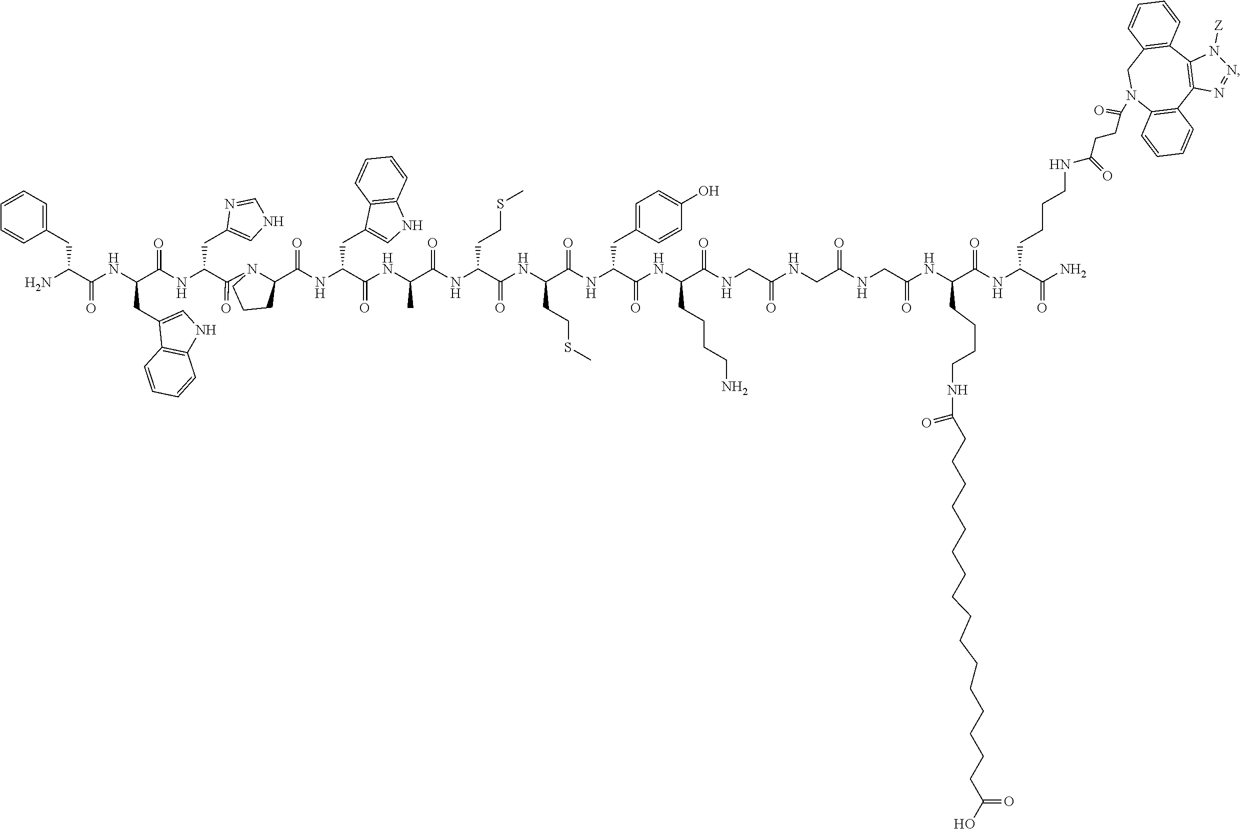

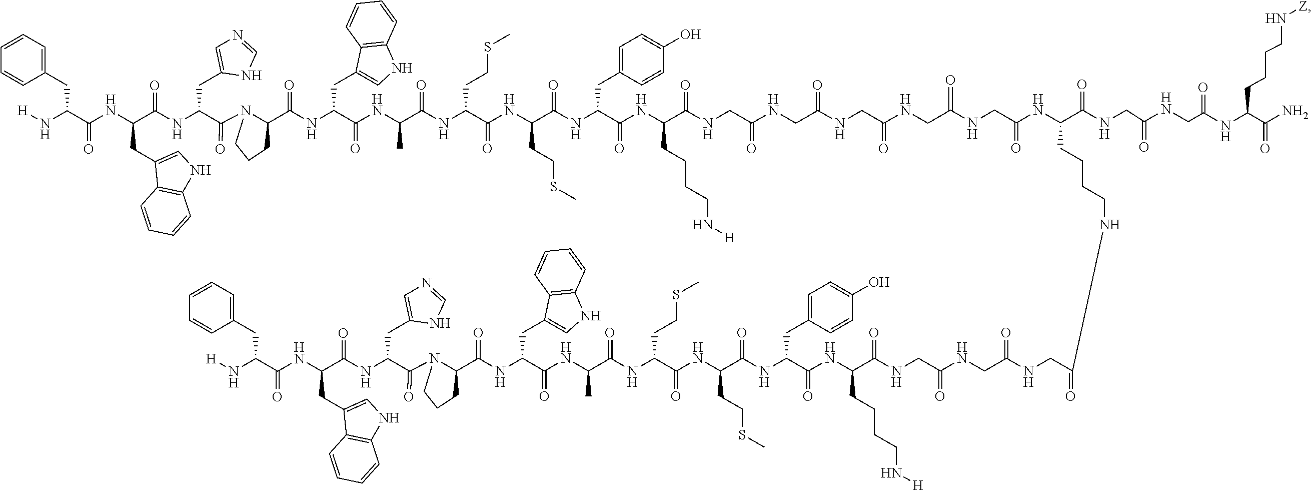

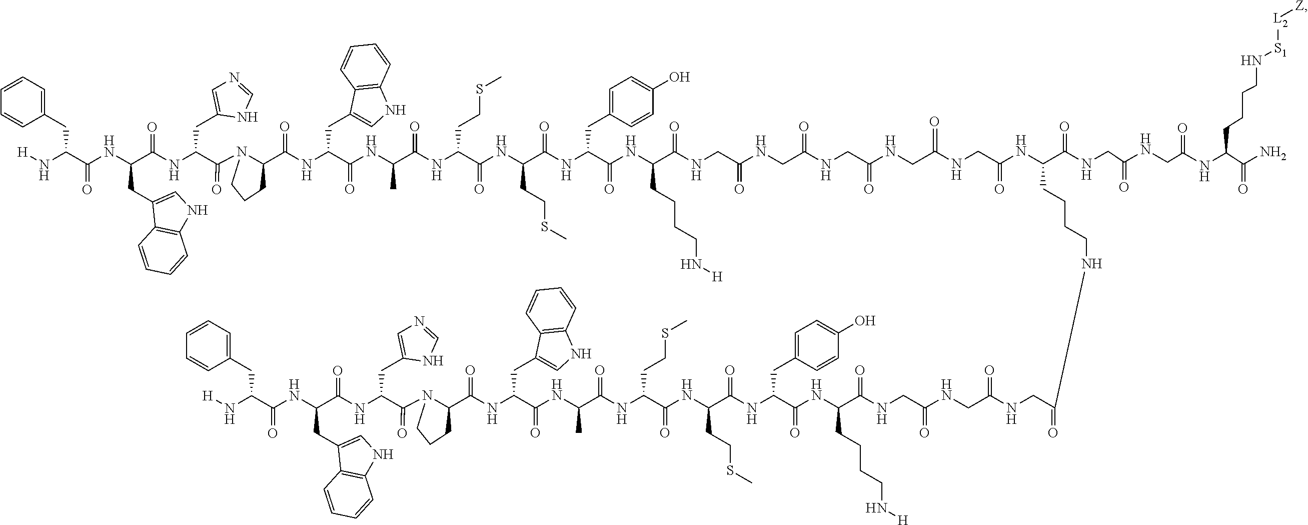

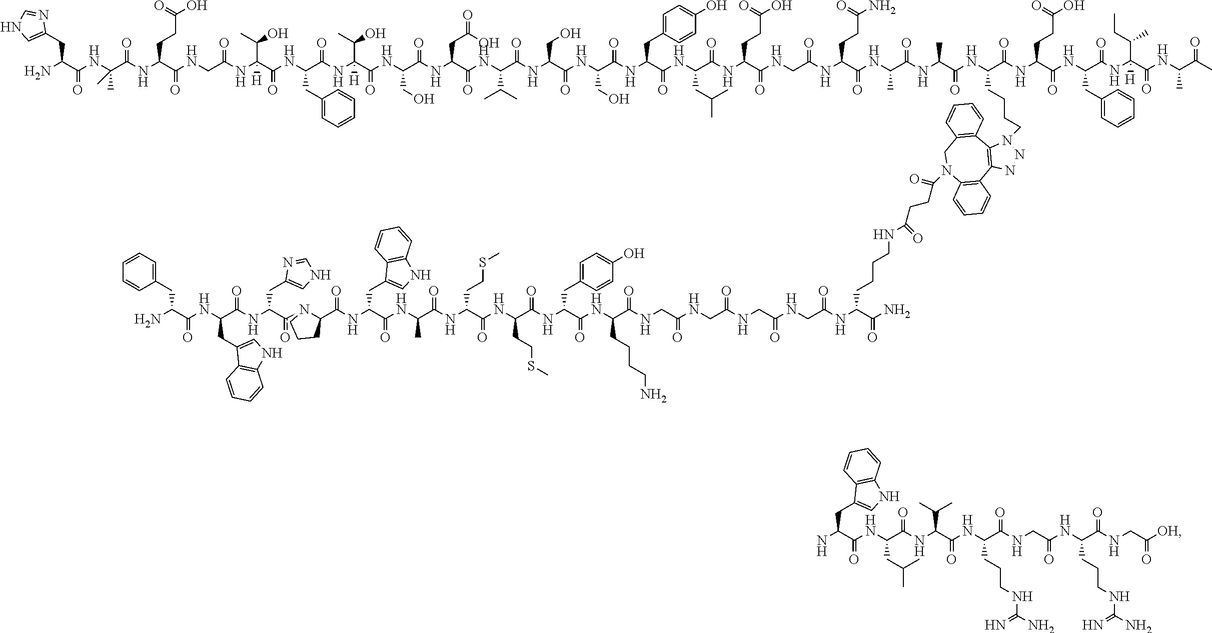

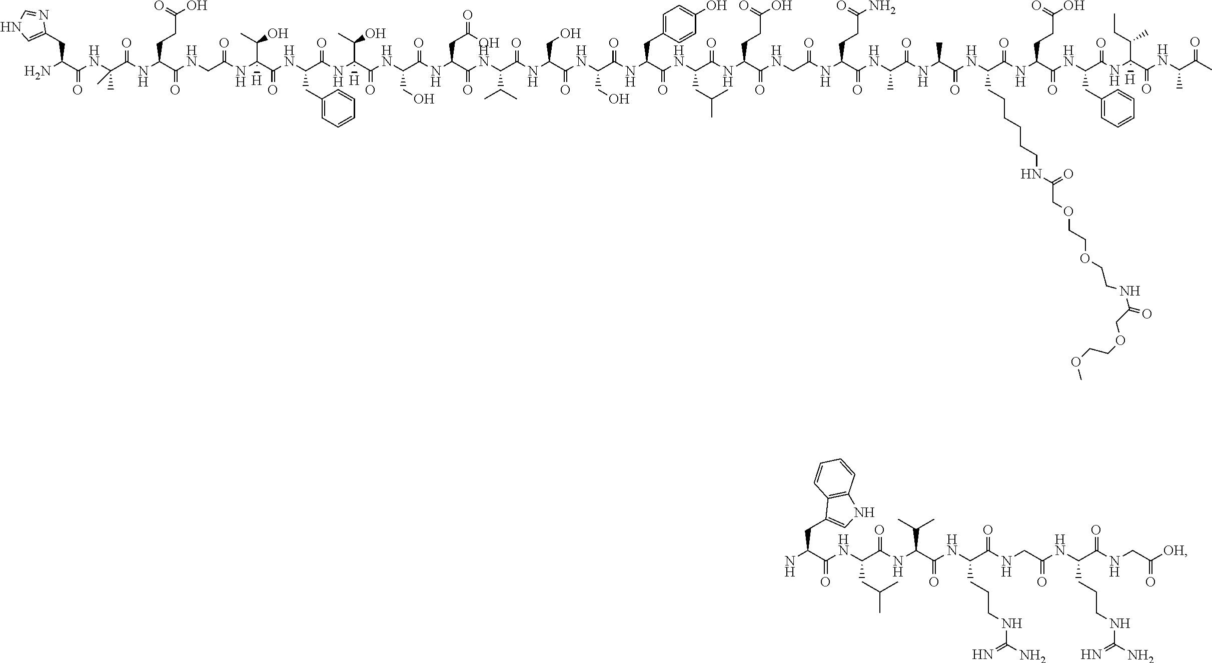

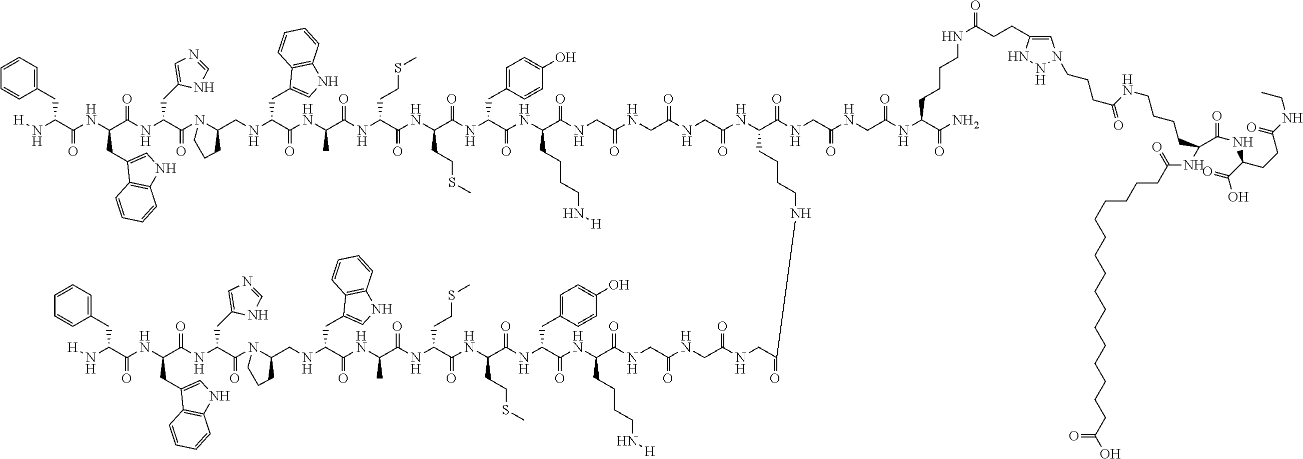

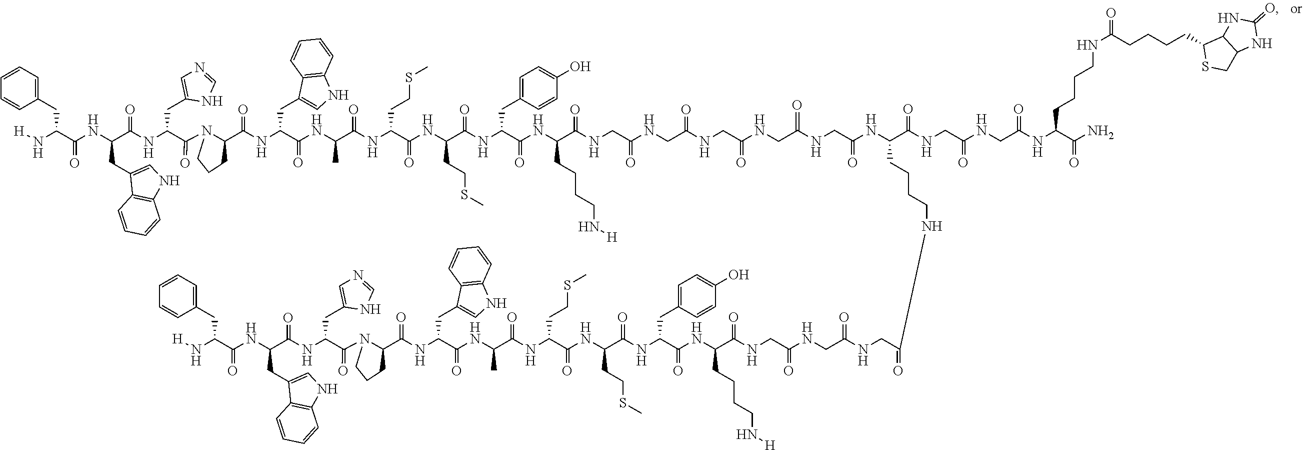

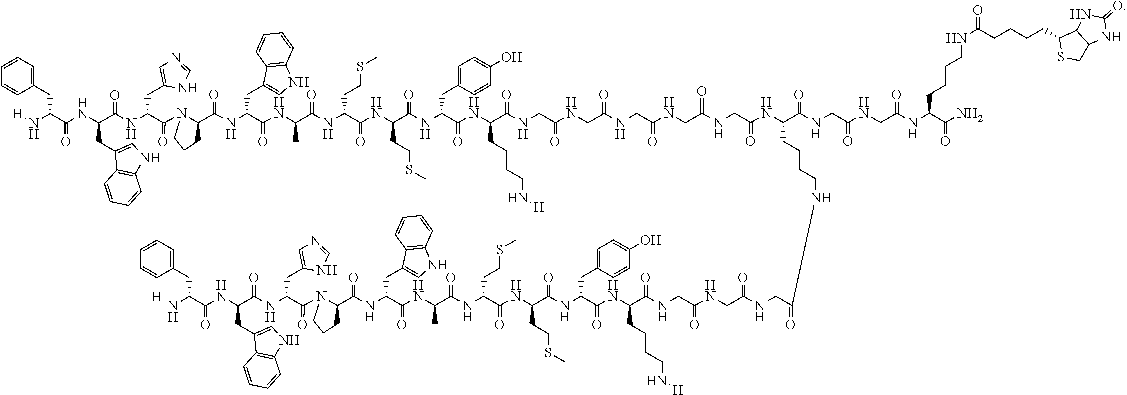

110-115. (canceled)

116. The conjugate of claim 30, wherein the conjugate has the structure: 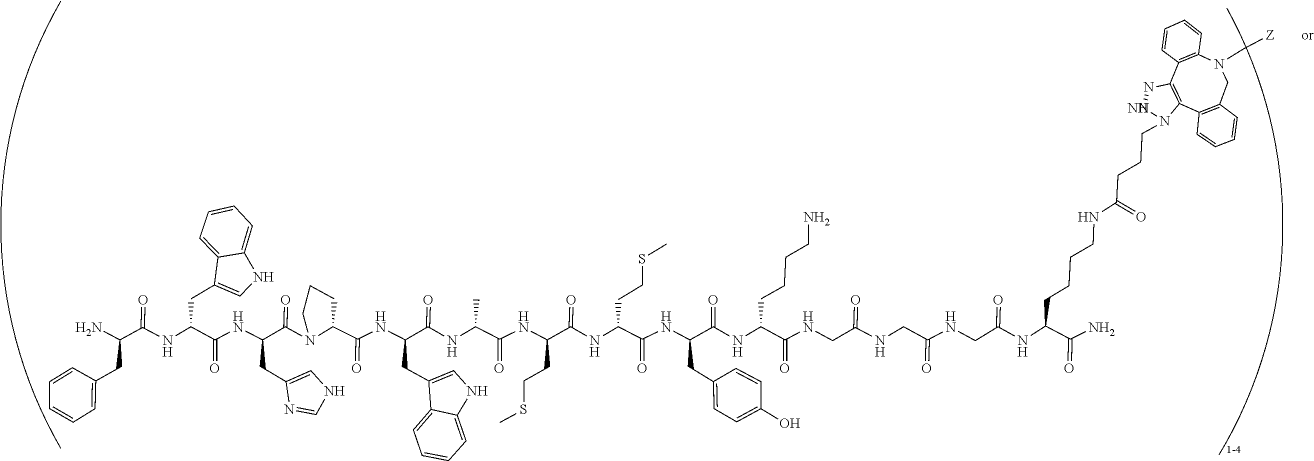 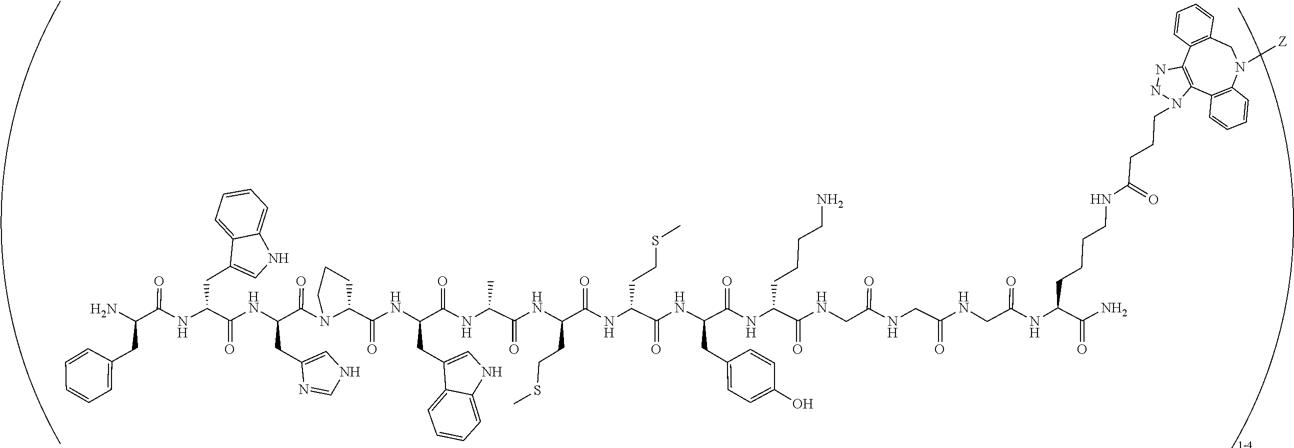
wherein Z is a therapeutic antibody, optionally wherein the therapeutic antibody is Adalimumab.

117. (canceled)

118. A pharmaceutical composition, comprising a conjugate of claim 1; and a pharmaceutical acceptable excipient.

119. A method of treating a disease, comprising administering to a subject in need thereof an effective amount of the conjugate of claim 1, wherein the active moiety is a drug moiety and the disease is the therapeutic target of the drug moiety, or
of treating a disease, comprising administering to a subject in need thereof an effective amount of the conjugate of claim 1, wherein the active moiety is a GLP-1 peptide variant and the disease is the therapeutic target of the GLP-1 peptide variant, or
of inducing antigen-specific immunotolerance, comprising administering to a subject in need thereof an effective amount of the conjugate of claim 1, wherein the active moiety is an erythrocyte-targeted antigen moiety.

120.-156. (canceled)
